(19) United States
(12) Patent Application Publication
(10) Pub. No.: US 2024/0392094 A1
(43) Pub. Date: Nov. 28, 2024

(54) Method for Screening Solvent for Polyvinyl Chloride Extraction, Recycling Method for Waste Material, and Recycled Polyvinyl Chloride and Composition

(71) Applicant: LG Chem, Ltd., Seoul (KR)

(72) Inventors: Kyeong Hwan Han, Daejeon (KR); Eunbyeol Ko, Daejeon (KR); Kyoung Hoon Kim, Daejeon (KR); Seungha Kim, Daejeon (KR); Hyung Sop Kim, Daejeon (KR); Gahyeon Lee, Daejeon (KR); Seung Yong Lee, Daejeon (KR); Yoonsun Jang, Daejeon (KR)

(73) Assignee: LG Chem, Ltd., Seoul (KR)

(21) Appl. No.: 18/688,127

(22) PCT Filed: Sep. 2, 2022
(86) PCT No.: PCT/KR2022/013222

§ 371 Date: Feb. 29, 2024

(30) Foreign Application Priority Data
Sep. 3, 2021 (KR) 10-2021-0117874
Sep. 3, 2021 (KR) 10-2021-0117881
Sep. 1, 2022 (KR) 102022-0110822

(51) Int. Cl. C08J 11/08; G16C 60/00

(52) U.S. Cl. CPC C08J 11/08; G16C 60/00; C08J 2327/06

(57) ABSTRACT

A method for screening a solvent for extracting polyvinyl chloride (PVC) from waste using the following Equation 1:

$$PVC solubility score = {x}_{1} exp ( - {x}_{2} {μ}_{pvc}^{sol} - {x}_{3} {μ}_{dop}^{sol} ) + {x}_{4} exp ( - {x}_{5} {r}_{pvc} - {x}_{6} {r}_{dop} ) - {x}_{7} exp ( - {x}_{8} {V}_{sol} ) + {x}_{9} ,$$

μᵢ^{j} is a chemical potential of the solvent, and in μᵢ^{j}, i is a solute and j is the solvent, rᵢ is a Hansen solubility parameter distance of the solvent, Vₛₒₗ is a molecular volume of the solvent, sol is the solvent, pvc is polyvinyl chloride, and dop is diethylhexyl phthalate. A method for recycling waste is also provided. Additionally, a recycled polyvinyl chloride and a recycled composition are provided.

### CROSS-REFERENCE TO RELATED APPLICATIONS

This application is a national phase entry under 35 U.S.C. § 371 of International Application No. PCT/KR2022/013222 filed on Sep. 2, 2022, which claims priority to Korean Patent Application Nos. 10-2021-0117881, 10-2021-0117874, and 10-2022-0110822 filed on Sep. 3, 2021, Sep. 3, 2021, and Sep. 1, 2022, respectively, the entire contents of which are incorporated herein by reference.

### TECHNICAL FIELD

The present specification relates to a method for screening a solvent for extracting polyvinyl chloride, a method for recycling waste, recycled polyvinyl chloride, and a recycled composition.

### BACKGROUND ART

Since plastic can be molded into various shapes by heat or pressure, and the quality of the material is getting better and better, things in everyday life are being replaced with plastic, and new things using the same are being developed.

Since plastics are convenient and easy to use, demand for products made of plastics, such as plastic bags, PET bottles, disposable items, packaging products, and electronic products, is gradually increasing all over the world.

Such used and discarded plastics are either incinerated or landfilled. In the case of incineration, many contaminants harmful to the human body are discharged, so it is preferrable that the discarded plastics are landfilled rather than incinerated.

However, it takes a long time to naturally decompose most plastics after disposal, and the amount of plastics that end up in landfills is substantial, so interest in recycling plastics is increasing.

Among plastics, polyvinyl chloride (PVC) has been widely used in building materials (pipes, window frames, and the like), electric wire coatings, synthetic leather, rubber tubs, hoses, flooring materials, packing materials, medical sap bags, toys, and the like. Various methods for recycling PVC from waste including PVC and materials capable of minimizing damage to humans and the environment during the recycling process need to be explored.

### Technical Problem

The present specification provides a method for screening a solvent for extracting polyvinyl chloride, a method for recycling waste, recycled polyvinyl chloride, and a recycled composition.

### Technical Solution

An exemplary embodiment of the present specification provides a method for screening a solvent for extracting polyvinyl chloride. The method includes screening a solvent for extracting polyvinyl chloride from waste including the polyvinyl chloride using the following Equation 1.

$$PVC solubility score = {x}_{1} exp ( - {x}_{2} {μ}_{pvc}^{sol} - {x}_{3} {μ}_{dop}^{sol} ) + {x}_{4} exp ( - {x}_{5} {r}_{pvc} - {x}_{6} {r}_{dop} ) - {x}_{7} exp ( - {x}_{8} {V}_{sol} ) + {x}_{9} \qquad [ Equation 1 ]$$

In Equation 1,
xₖ is each a real number from −20 to 20, and k is an integer from 1 to 9,
μᵢ^{j} is the chemical potential of a solvent, and in μᵢ^{j}, is a solute and j is a solvent,
rᵢ is the Hansen solubility parameter distance of the solvent,
Vₛₒₗ is the molecular volume of the solvent, and
sol is the solvent, pvc is polyvinyl chloride, and dop is diethylhexyl phthalate.

Another exemplary embodiment of the present specification provides a method for recycling waste including polyvinyl chloride. The method includes screening a solvent for extracting polyvinyl chloride using the above-described method for screening the solvent, and extracting polyvinyl chloride from waste including the polyvinyl chloride using the screened solvent.

Still another exemplary embodiment of the present specification provides recycled polyvinyl chloride extracted from waste including polyvinyl chloride using a solvent screened by the above-described method for screening the solvent.

Yet another exemplary embodiment of the present specification provides a waste polyvinyl chloride recycled composition including polyvinyl chloride extracted from waste including polyvinyl chloride, two or more good solvents of solvents with a PVC solubility score value of 11 or more according to the following Equation 1, and at least one non-solvent of solvents with a PVC solubility score value of less than 11 according to the following Equation 1.

$$PVC solubility score = {x}_{1} exp ( - {x}_{2} {μ}_{pvc}^{sol} - {x}_{3} {μ}_{dop}^{sol} ) + {x}_{4} exp ( - {x}_{5} {r}_{pvc} - {x}_{6} {r}_{dop} ) - {x}_{7} exp ( - {x}_{8} {V}_{sol} ) + {x}_{9} \qquad [ Equation 1 ]$$

In Equation 1,
xₖ is each a real number from −20 to 20, and k is an integer from 1 to 9,
μᵢ^{j} is the chemical potential of a solvent, and in μᵢ^{j}, is a solute and j is a solvent,
rᵢ is the Hansen solubility parameter distance of the solvent,
Vₛₒₗ is the molecular volume of the solvent, and
sol is the solvent, pvc is polyvinyl chloride, and dop is diethylhexyl phthalate.

### Advantageous Effects

According to an exemplary embodiment of the present specification, it is possible to find various solvents capable of being used in the process of extracting polyvinyl chloride from waste including polyvinyl chloride.

According to another exemplary embodiment of the present specification, it is possible to find various good solvent-non-solvent combinations suitable for the process of extracting polyvinyl chloride from waste including polyvinyl chloride.

### BRIEF DESCRIPTION OF DRAWINGS

FIG. 1 is a graph showing PVC solubility score values calculated by Equation 1 according to the present specification.

FIG. 2 is a graph showing the relationship between the PVC solubility score value calculated by Equation 1 according to the present specification and the PVC swelling ratio.

### DETAILED DESCRIPTION

Hereinafter, the present specification will be described in detail.

The present specification has been made in an effort to provide a parameter capable of classifying and evaluating a solvent available for extracting polyvinyl chloride (PVC) from waste by predicting the solvent. Through this, a new solvent capable of extracting PVC may be discovered. Here, the solvent capable of extracting PVC means all solvents used for extracting PVC from waste, and specifically, may include not only a good solvent that has high solubility in PVC and thus can dissolve PVC from waste, but also a non-solvent that can obtain PVC by precipitating PVC from a good solvent in which PVC is dissolved. Further, one or more solvents can be mixed and used for one role. For example, a single solvent may be used as a good solvent, and a mixture of two or more solvents may be used as the good solvent, and in this case, in consideration of a mixing ratio of each solvent in a mixed solvent, that is, a weight fraction, a PVC solubility score value is calculated by substituting the weight fraction into Equation 1 to be described below.

An exemplary embodiment of the present specification provides a method for screening a solvent for extracting polyvinyl chloride, the method including screening a solvent for extracting polyvinyl chloride from waste including the polyvinyl chloride using the following Equation 1.

$$PVC solubility score = {x}_{1} exp ( - {x}_{2} {μ}_{pvc}^{sol} - {x}_{3} {μ}_{dop}^{sol} ) + {x}_{4} exp ( - {x}_{5} {r}_{pvc} - {x}_{6} {r}_{dop} ) - {x}_{7} exp ( - {x}_{8} {V}_{sol} ) + {x}_{9} \qquad [ Equation 1 ]$$

In Equation 1,
xₖ is each a real number from −20 to 20, and k is an integer from 1 to 9,
μᵢ^{j} is the chemical potential of a solvent, and in μᵢ^{j}, i is a solute and j is a solvent,
rᵢ is the Hansen solubility parameter distance of the solvent,
Vₛₒₗ is the molecular volume of the solvent, and
sol is the solvent, pvc is polyvinyl chloride, and dop is diethylhexyl phthalate.

In an exemplary embodiment of the present specification, the affinity between PVC and a solvent is calculated using data such as the chemical potential of the solvent, the Hansen solubility parameter distance rᵢ of the solvent, and the solvent molecular volume Vₛₒₗ, and based on the calculated results, it is possible to classify a good solvent that mixes well with PVC, that is, a solvent capable of separating PVC from waste, and a non-solvent that does not mix well with PVC, that is, a solvent capable of precipitating PVC from a solvent in which PVC is dissolved.

In an exemplary embodiment of the present specification, Equation 1 means a solubility score in consideration of factors capable of determining the solubility between a PVC including a plasticizer and a solvent along with the variables of experimental conditions such as temperature. Since Equation 1 exhibits a tendency that the higher the solubility in PVC, the higher the value, the higher the value, the more suitable the solvent is for use as a solvent for dissolving PVC, and the lower the value, the more suitable the solvent is for use as a non-solvent.

In an exemplary embodiment of the present specification, through the value of Equation 1, a good solvent and a non-solvent may be distinguished based on a reference value for distinguishing the good solvent and the non-solvent of 8, 8.5, 9, 9.5, 10, 10.5, 11, 11.5, 12, 12.5, 13, 13.5, 14, 14.5, 15, or 15.5. Specifically, when the value is the reference value or more, the solvent may be determined as a good solvent, and when the value is less than the reference value, the solvent may be determined as a non-solvent.

An exemplary embodiment of the present specification may select two or more of solvents with a PVC solubility score value of 8 or more according to Equation 1 as a good solvent for extracting polyvinyl chloride from waste including the polyvinyl chloride and may select at least one of solvents with a PVC solubility score value of less than 8 according to Equation 1 as a non-solvent.

An exemplary embodiment of the present specification may select two or more of solvents with a PVC solubility score value of 8.5 or more according to Equation 1 as a good solvent for extracting polyvinyl chloride from waste including the polyvinyl chloride and may select at least one of solvents with a PVC solubility score value of less than 8.5 or 8 or less according to Equation 1 as a non-solvent.

An exemplary embodiment of the present specification may select two or more of solvents with a PVC solubility score value of 9 or more according to Equation 1 as a good solvent for extracting polyvinyl chloride from waste including the polyvinyl chloride and may select at least one of solvents with a PVC solubility score value of less than 9, 8.5 or less, or 8 or less according to Equation 1 as a non-solvent.

An exemplary embodiment of the present specification may select two or more of solvents with a PVC solubility score value of 9.5 or more according to Equation 1 as a good solvent for extracting polyvinyl chloride from waste including the polyvinyl chloride and may select at least one of solvents with a PVC solubility score value of less than 9.5, 9 or less, 8.5 or less, or 8 or less according to Equation 1 as a non-solvent.

An exemplary embodiment of the present specification may select two or more of solvents with a PVC solubility score value of 10 or more according to Equation 1 as a good solvent for extracting polyvinyl chloride from waste including the polyvinyl chloride and may select at least one of solvents with a PVC solubility score value of less than 10, 9.5 or less, 9 or less, 8.5 or less, or 8 or less according to Equation 1 as a non-solvent.

An exemplary embodiment of the present specification may select two or more of solvents with a PVC solubility score value of 10.5 or more according to Equation 1 as a good solvent for extracting polyvinyl chloride from waste including the polyvinyl chloride and may select at least one of solvents with a PVC solubility score value of less than 10.5, 10 or less, 9.5 or less, 9 or less, 8.5 or less, or 8 or less according to Equation 1 as a non-solvent.

An exemplary embodiment of the present specification may select two or more of solvents with a PVC solubility score value of 11 or more according to Equation 1 as a good solvent for extracting polyvinyl chloride from waste including the polyvinyl chloride and may select at least one of solvents with a PVC solubility score value of less than 11, 10.5 or less, 10 or less, 9.5 or less, 9 or less, 8.5 or less, or 8 or less according to Equation 1 as a non-solvent.

An exemplary embodiment of the present specification may select two or more of solvents with a PVC solubility score value of 11.5 or more according to Equation 1 as a good solvent for extracting polyvinyl chloride from waste including the polyvinyl chloride and may select at least one of solvents with a PVC solubility score value of less than 11.5, 11 or less, 10.5 or less, 10 or less, 9.5 or less, 9 or less, 8.5 or less, or 8 or less according to Equation 1 as a non-solvent.

An exemplary embodiment of the present specification may select two or more of solvents with a PVC solubility score value of 12 or more according to Equation 1 as a good solvent for extracting polyvinyl chloride from waste including the polyvinyl chloride and may select at least one of solvents with a PVC solubility score value of less than 12, 11.5 or less, 11 or less, 10.5 or less, 10 or less, 9.5 or less, 9 or less, 8.5 or less, or 8 or less according to Equation 1 as a non-solvent.

An exemplary embodiment of the present specification may select two or more of solvents with a PVC solubility score value of 12.5 or more according to Equation 1 as a good solvent for extracting polyvinyl chloride from waste including the polyvinyl chloride and may select at least one of solvents with a PVC solubility score value of less than 12.5, 12 or less, 11.5 or less, 11 or less, 10.5 or less, 10 or less, 9.5 or less, 9 or less, 8.5 or less, or 8 or less according to Equation 1 as a non-solvent.

An exemplary embodiment of the present specification may select two or more of solvents with a PVC solubility score value of 13 or more according to Equation 1 as a good solvent for extracting polyvinyl chloride from waste including the polyvinyl chloride and may select at least one of solvents with a PVC solubility score value of less than 13, 12.5 or less, 12 or less, 11.5 or less, 11 or less, 10.5 or less, 10 or less, 9.5 or less, 9 or less, 8.5 or less, or 8 or less according to Equation 1 as a non-solvent.

An exemplary embodiment of the present specification may select two or more of solvents with a PVC solubility score value of 11 or more according to Equation 1 as a good solvent for extracting polyvinyl chloride from waste including the polyvinyl chloride and may select at least one of solvents with a PVC solubility score value of less than 11 according to Equation 1 as a non-solvent.

In Equation 1 of an exemplary embodiment of the present specification, μᵢ^{j} is the chemical potential of the solvent at room temperature, and is the chemical potential when one solute molecule is weakly dissolved in the solvent. The chemical potential is expressed as μ, i is the solute, j is the solvent, where sol is the solvent, pvc is polyvinyl chloride, and dop is diethylhexyl phthalate. Specifically, μ_{pvc}^{sol} means the chemical potential when one molecule of PVC is included in an infinite number of solvents at room temperature, and is a value that indicates how much affinity PVC has with solvents. μ_{dop}^{sol} means the chemical potential when one molecule of a plasticizer is included in an infinite number of solvents at room temperature, and is a value that indicates affinity between the plasticizer and the solvent. A smaller value of μᵢ^{j} indicates a higher affinity. In this case, normal temperature means natural atmospheric temperature without heating or cooling, and is, for example, 20±5° C.

In an exemplary embodiment of the present specification, the aforementioned diethylhexyl phthalate is the most common plasticizer used in waste polyvinyl chloride, and has chemically similar properties even when different plasticizers are used, and diethylhexyl phthalate showing representativeness was selected.

In addition, about 20 wt % to 50 wt % of a plasticizer is introduced based on the total weight of the final product during the preparation of polyvinyl chloride. In order to extract PVC from waste, it is preferred to consider the properties of the plasticizer added in a non-negligible content, and when PVC is extracted from waste, there is an advantage in that more PVC can be extracted when the solubility with a plasticizer is high, so factors related to the interaction between plasticizer and solvent are included in Equation 1.

In Equation 1 of an exemplary embodiment of the present specification, the chemical potential can be calculated using a Conductor like Screening Model for Real Solvents (COSMO-RS) theory, and COSMO-RS predicts thermodynamic data, which are properties of each solvent, with molecular structure information based on unimolecular quantum chemical calculations. The chemical potential can be calculated using the COSMOtherm software (COSMOlogic GmbH & Co. KG), which can conveniently calculate the chemical potential.

In Equation 1 of an exemplary embodiment of the present specification, rᵢ is the Hansen solubility parameter distance between the solvent and the solute i, and is calculated by the following Equation 2. r_{pvc} is the Hansen solubility parameter distance between the solvent and PVC, and indicates how similar PVC and the solvent are r_{dop} is the Hansen solubility parameter distance between the solvent and the plasticizer, and indicates how similar the plasticizer and the solvent are. It is determined that the more similar properties are, the higher the compatibility is, and it is determined that the smaller the rᵢ, the more similar the plasticizer and the solvent are.

$${r}_{i} = {( 4 \times {( d {D}_{i} - d {D}_{sol} )}^{2} + {( d {P}_{i} - d {P}_{sol} )}^{2} + {( d {H}_{i} - d {H}_{sol} )}^{2} )}^{1 / 2} \qquad [ Equation 2 ]$$

In Equation 2, dDᵢ-dDₛₒₗ is the diffusion energy difference between molecules of solute i and solvent,
dPᵢ-dPₛₒₗ is the dipole interaction energy difference between molecules of solute i and solvent, and
dHᵢ-dHₛₒₗ is the hydrogen bonding energy difference between molecules of solute i and solvent.

In Equation 1 of an exemplary embodiment of the present specification, the Hansen solubility parameter can be calculated by Yamamoto Molecule Breaking (Y-MB) and can be calculated using the HSPip software which conveniently calculates the Hansen solubility parameter. Specifically, molecules are separated into sub-groups using HSPip software to calculate the Hansen solubility parameter by a neural network method in consideration of the number corresponding to each sub-group and the inter-group interaction.

In Equation 1 of an exemplary embodiment of the present specification, Vₛₒₗ is the molecular volume of the solvent, and the smaller the volume of the solvent molecule, the higher the probability of penetrating well the space between polymers and dissolving PVC, so a relevant factor is included.

In Equation 1 of an exemplary embodiment of the present specification, the molecular volume of the solvent can be calculated by quantitative-structure-property-relationship (QSPR), and can be calculated using the COSMOtherm software (COSMOlogic GmbH & Co. KG [symbol] ) which conveniently calculates the molecular volume of the solvent.

In an exemplary embodiment of the present specification, xₖ is each a real number from −20 to 20, and k is an integer from 1 to 9. Specifically, x₁ to x₉ are each a real number from −20 to 20.

In an exemplary embodiment of the present specification, x₁ is a control variable that indicates the degree of effect of the chemical potential obtained by COSMOtherm among the solubility prediction methods of PVC and a plasticizer on a solvent. x₁ is a real number from −20 to 20, specifically a real number from −10 to 10, and more specifically a real number from 0 to 5, and may be preferably 1.

In an exemplary embodiment of the present specification, x₂ is a control variable to consider the degree of effect of the chemical potential of PVC on the solvent. In this case, for x₂, the corresponding variable is adjusted according to the content of the plasticizer included in PVC and process conditions (temperature, pressure, time, and the like). x₂ is a real number from −20 to 20, specifically a real number from −10 to 10, and more specifically a real number from −5 to 5, and may be preferably 0.05.

In an exemplary embodiment of the present specification, x₃ is a control variable to consider the degree of effect of the chemical potential of the plasticizer on the solvent. In this case, for x₃, the corresponding variable is adjusted according to the content of the plasticizer included in PVC and process conditions (temperature, pressure, time, and the like). x₃ is a real number from −20 to 20, specifically a real number from −10 to 10, and more specifically a real number from −5 to 5, and may be preferably 0.04.

In an exemplary embodiment of the present specification, x₄ is a control variable that indicates the degree of effect of the Hansen solubility parameter distance among the solubility prediction methods of PVC and the plasticizer on the solvent. x₄ is a real number from −20 to 20, specifically a real number from 0 to 20, and more specifically a real number from 5 to 15, and may be preferably 10.

In an exemplary embodiment of the present specification, x₅ is a control variable to consider the degree of effect of the Hansen solubility parameter distance of PVC on the solvent. In this case, for x₅, the corresponding variable is adjusted according to the content of the plasticizer included in PVC and process conditions (temperature, pressure, time, and the like). x₅ is a real number from −20 to 20, specifically a real number from −10 to 10, and more specifically a real number from −5 to 5, and may be preferably 0.03.

In an exemplary embodiment of the present specification, x₆ is a control variable to consider the degree of effect of the Hansen solubility parameter distance of the plasticizer on the solvent. In this case, for x₆, the corresponding variable is adjusted according to the content of the plasticizer included in PVC and process conditions (temperature, pressure, time, and the like). x₆ is a real number from −20 to 20, specifically a real number from −10 to 10, and more specifically a real number from −5 to 5, and may be preferably 0.003.

In an exemplary embodiment of the present specification, x₇ is a control variable to consider the influence of the solvent molecular volume. Since increasing process temperature has less effect on molecular volume, x₇ may be decreased, and the effect according to PVC composition and molecular weight may be adjusted. x₇ is a real number from −20 to 20, specifically a real number from −10 to 10, and more specifically a real number from −5 to 5, and may be preferably 0.001.

In an exemplary embodiment of the present specification, x₈ is a control variable to consider the effect of the solvent molecular volume. In this case, x₈ may adjust the effect according to the PVC composition and molecular weight. x₈ is a real number from −20 to 20, specifically a real number from −10 to 10, and more specifically a real number from −5 to 5, and may be preferably 0.0001.

In an exemplary embodiment of the present specification, x₉ is a control variable to regulate the reference of the PVC solubility score value. x₉ is a real number from −20 to 20, specifically a real number from −10 to 10, and more specifically a real number from −5 to 5, and may be preferably 0.

In an exemplary embodiment of the present specification, parameters of the control variables are adjusted according to the content of the plasticizer included in the wasted PVC and process conditions (temperature, pressure, time, and the like). For example, when the plasticizer content is low, x₂ and x₅ are increased and x₃ and x₆ are decreased. Further, when the process temperature increases, the effect on the molecular volume is reduced, so x₇ is decreased, and x₁ and x₄ are increased as the temperature rises in order to correct the chemical potential and HSP distance calculated based on room temperature.

In an exemplary embodiment of the present specification, a solvent with a PVC solubility score value of 13 or more according to Equation 1 may be determined to be a good solvent for polyvinyl chloride. In this case, the good solvent means a solvent capable of dissolving polyvinyl chloride, and is a solvent capable of dissolving and extracting polyvinyl chloride from the waste.

In an exemplary embodiment of the present specification, when x₁ is 1, x₂ is 0.005, x₃ is 0.04, x₄ is 10, x₅ is 0.03, x₆ is 0.003, x₇ is 0.001, x₈ is 0.0001, and x₉ is 0, a solvent with a PVC solubility score value of 13 or more according to Equation 1 may be determined to be a good solvent for polyvinyl chloride.

In an exemplary embodiment of the present specification, a solvent with a PVC solubility score value of less than 13 according to Equation 1 may be determined to be a non-solvent for polyvinyl chloride. In this case, the non-solvent means a solvent that has low solubility in polyvinyl chloride, and is a solvent that can be used when polyvinyl chloride is precipitated from an extracted polyvinyl chloride solution.

In an exemplary embodiment of the present specification, when x₁ is 1, x₂ is 0.005, x₃ is 0.04, x₄ is 10, x₅ is 0.03, x₆ is 0.003, x₇ is 0.001, x₈ is 0.0001, and x₉ is 0, a solvent with a PVC solubility score value of less than 13 according to Equation 1 may be determined to be a non-solvent for polyvinyl chloride.

Another exemplary embodiment of the present specification provides a method for screening a solvent for extracting polyvinyl chloride. The method includes selecting two or more of solvents with a PVC solubility score value of 11 or more according to Equation 1 as a good solvent for extracting polyvinyl chloride from waste including the polyvinyl chloride, and selecting at least one of solvents with a PVC solubility score value of less than 11 according to Equation 1 as a non-solvent for extracting polyvinyl chloride from waste including the polyvinyl chloride.

Still another exemplary embodiment of the present specification provides a method for screening a solvent for extracting polyvinyl chloride. The method includes selecting two or more of solvents with a PVC solubility score value of 13 or more according to Equation 1 as a good solvent for extracting polyvinyl chloride from waste including the polyvinyl chloride, and selecting at least one of solvents with a PVC solubility score value of 9 or less according to Equation 1 as a non-solvent for extracting polyvinyl chloride from waste including the polyvinyl chloride.

In an exemplary embodiment of the present specification, a difference between the PVC solubility score value of the good solvent and the PVC solubility score value of the non-solvent may be 0.1 or more, 0.2 or more, 0.3 or more, 0.4 or more, 0.5 or more, 0.6 or more, 0.7 or more, 0.8 or more, 0.9 or more, 1 or more, 1.1 or more, 1.2 or more, 1.3 or more, 1.4 or more, 1.5 or more, 1.6 or more, 1.7 or more, 1.8 or more, 1.9 or more, 2 or more, 2.1 or more, 2.2 or more, 2.3 or more, 2.4 or more, 2.5 or more, 2.6 or more, 2.7 or more, 2.8 or more, 2.9 or more, 3 or more, 3.1 or more, 3.2 or more, 3.3 or more, 3.4 or more, 3.5 or more, 3.6 or more, 3.7 or more, 3.8 or more, 3.9 or more, 4 or more, 4.1 or more, 4.2 or more, 4.3 or more, 4.4 or more, 4.5 or more, 4.6 or more, 4.7 or more, 4.8 or more, 4.9 or more, 5 or more, 5.1 or more, 5.2 or more, 5.3 or more, 5.4 or more, 5.5 or more, 5.6 or more, 5.7 or more, 5.8 or more, 5.9 or more, 6 or more, 6.1 or more, 6.2 or more, 6.3 or more, 6.4 or more, 6.5 or more, 6.6 or more, 6.7 or more, 6.8 or more, 6.9 or more, 7 or more, 7.1 or more, 7.2 or more, 7.3 or more, 7.4 or more, 7.5 or more, 7.6 or more, 7.7 or more, 7.8 or more, 7.9 or more, 8 or more, 8.1 or more, 8.2 or more, 8.3 or more, 8.4 or more, 8.5 or more, 8.6 or more, 8.7 or more, 8.8 or more, 8.9 or more, 9 or more, 9.1 or more, 9.2 or more, 9.3 or more, 9.4 or more, 9.5 or more, 9.6 or more, 9.7 or more, 9.8 or more, 9.9 or more, or 10 or more.

In an exemplary embodiment of the present specification, the good solvent may have a boiling point of 70° C. or more, 71° C. or more, 72° C. or more, 73° C. or more, 74° C. or more, 75° C. or more, 76° C. or more, 77° C. or more, 78° C. or more, 79° C. or more, 80° C. or more, 81° C. or more, 82° C. or more, 83° C. or more, 84° C. or more, 85° C. or more, 86° C. or more, 87° C. or more, 88° C. or more, 89° C. or more, 90° C. or more, 95° C. or more, or 100° C. or more. In this case, when heating is required in the process, the good solvent has a boiling point higher than the heating temperature, so it is advantageous because there is no loss of good solvent due to heating, and the solvent is easily treated because the volatility is adjusted to some extent. In this case, the upper limit of the boiling point of the good solvent is not particularly limited, but when the boiling point is too high, the energy required for drying may increase during the drying process after the extraction of PVC is completed, so a good solvent may be selected in consideration of process costs. For example, the good solvent may have a boiling point of 400° C. or less, 350° C. or less, or 300° C. or less.

Another exemplary embodiment of the present specification provides a method for recycling waste including polyvinyl chloride, the method including screening a solvent for extracting polyvinyl chloride using the above-described method for screening the solvent, and extracting polyvinyl chloride from waste including the polyvinyl chloride using the screened solvent.

In the present specification, the description of the screening step in the waste recycling method may reference the above-described method for screening a solvent for extracting PVC.

In an exemplary embodiment of the present specification, the extracting of the polyvinyl chloride may include a dissolution process of bringing waste including the polyvinyl chloride into contact with a good solvent with a PVC solubility score value of 11 or more, specifically, 13 or more according to Equation 1 and obtaining a polyvinyl chloride solution by dissolving polyvinyl chloride in the waste in the good solvent.

In an exemplary embodiment of the present specification, the extracting of the polyvinyl chloride may include a dissolution process of bringing waste including the polyvinyl chloride into contact with two or more good solvents of solvents with a PVC solubility score value of 11 or more according to Equation 1 and obtaining a polyvinyl chloride solution by dissolving polyvinyl chloride in the waste in the good solvent, and a precipitation process of precipitating polyvinyl chloride from the solution by bringing the solution into contact with at least one non-solvent of solvents with a PVC solubility score value of less than 11 according to Equation 1.

In an exemplary embodiment of the present specification, the extracting of the polyvinyl chloride may include a dissolution process of bringing waste including the polyvinyl chloride into contact with two or more good solvents of solvents with a PVC solubility score value of 13 or more according to Equation 1 and obtaining a polyvinyl chloride solution by dissolving polyvinyl chloride in the waste in the good solvent, and a precipitation process of precipitating polyvinyl chloride from the solution by bringing the solution into contact with at least one non-solvent of solvents with a PVC solubility score value of 9 or less according to Equation 1.

In an exemplary embodiment of the present specification, the extracting of the polyvinyl chloride may further include a separation process of separating a solid material from a polyvinyl chloride solution obtained by the dissolution process. In this case, the solid material is a solid that is not dissolved in a good solvent of polyvinyl chloride, and corresponds to impurities.

In an exemplary embodiment of the present specification, the extracting of the polyvinyl chloride may further include a rinsing process of rinsing polyvinyl chloride precipitated by the precipitation process.

In an exemplary embodiment of the present specification, the extracting of the polyvinyl chloride may further include a drying process of drying polyvinyl chloride precipitated by the precipitation process.

Still another exemplary embodiment of the present specification provides recycled polyvinyl chloride extracted from waste including polyvinyl chloride using a solvent screened by the above-described method for screening the solvent.

Yet another exemplary embodiment of the present specification provides a waste polyvinyl chloride recycled composition including polyvinyl chloride extracted from waste including polyvinyl chloride, two or more good solvents of solvents with a PVC solubility score value of 11 or more according to the following Equation 1, and at least one non-solvent of solvents with a PVC solubility score value of less than 11 according to the following Equation 1.

$$PVC solubility score = {x}_{1} exp ( - {x}_{2} {μ}_{pvc}^{sol} - {x}_{3} {μ}_{dop}^{sol} ) + {x}_{4} exp ( - {x}_{5} {r}_{pvc} - {x}_{6} {r}_{dop} ) - {x}_{7} exp ( - {x}_{8} {V}_{sol} ) + {x}_{9} \qquad [ Equation 1 ]$$

In Equation 1,
xₖ is each a real number from −20 to 20, and k is an integer from 1 to 9,
μᵢ^{j} is the chemical potential of a solvent, and in μᵢ^{j} is a solute and j is a solvent,
rᵢ is the Hansen solubility parameter distance of the solvent,
Vₛₒₗ is the molecular volume of the solvent, and
sol is the solvent, pvc is polyvinyl chloride, and dop is diethylhexyl phthalate.

Here, for the description on Equation 1, the above-described description is referenced to avoid duplication.

In an exemplary embodiment of the present specification, a waste polyvinyl chloride recycled composition means a state including all of the polyvinyl chloride extracted by the good solvent from waste including polyvinyl chloride, a good solvent used during the extraction of polyvinyl chloride, and a non-solvent that separates and precipitates the extracted polyvinyl chloride from the good solvent. For polyvinyl chloride extracted according to the ratio of the good solvent and the non-solvent, polyvinyl chloride dissolved in the good solvent and precipitated polyvinyl chloride may coexist, or most of polyvinyl chloride may be precipitated and settled in a solid state.

In an exemplary embodiment of the present specification, when the weight of the good solvent in the waste polyvinyl chloride recycled composition is set to 100, the content of the non-solvent is at least 100 parts by weight, and the solute may be precipitated by adding the non-solvent in an amount, which is equal to or more than the weight of the good solvent. The higher the content of non-solvent, the easier it is to precipitate the solute and the process time can be shortened, but in order to increase the purity in the solute, it is preferred to adjust the content of the non-solvent in consideration of the appropriate precipitation rate and the particle size of the precipitated solute.

In an exemplary embodiment of the present specification, when the weight of the good solvent is set to 100, the content of the non-solvent may be 100 parts by weight or more, 150 parts by weight or more, 200 parts by weight or more, 250 parts by weight or more, 300 parts by weight or more, 350 parts by weight or more, 400 parts by weight or more, 450 parts by weight or more or 500 parts by weight or more, 1000 parts by weight or less, 900 parts by weight or less, 800 parts by weight or less, 700 parts by weight or less, or 600 parts by weight or less.

In an exemplary embodiment of the present specification, the content of the good solvent is not particularly limited as long as the PVC in the waste can be dissolved. When the weight of the waste including waste polyvinyl chloride is set to 100, the content of the good solvent is at least 100 parts by weight, and the solute may be precipitated by adding the good solvent in an amount, which is equal to or more than the weight of the waste.

In an exemplary embodiment of the present specification, when the weight of the waste including waste polyvinyl chloride is set to 100, the content of the good solvent may be 100 parts by weight or more, 150 parts by weight or more, 200 parts by weight or more, 250 parts by weight or more, 300 parts by weight or more, 350 parts by weight or more, 400 parts by weight or more, 450 parts by weight or more or 500 parts by weight or more, 1000 parts by weight or less, 900 parts by weight or less, 800 parts by weight or less, 700 parts by weight or less, or 600 parts by weight or less.

In an exemplary embodiment of the present specification, the good solvent and the non-solvent are each independently any one of an alcohol-based solvent, a ketone-based solvent, an ester-based solvent, an ether-based solvent, a polar nonprotic solvent, a hydrocarbon solvent, or a halogen-containing solvent.

In an exemplary embodiment of the present specification, the good solvent is any one of an alcohol-based solvent, a ketone-based solvent, an ester-based solvent, an ether-based solvent, a polar nonprotic solvent, a hydrocarbon solvent, or a halogen-containing solvent.

In an exemplary embodiment of the present specification, the good solvent may include a main solvent present in an amount of 50 wt % or more in the good solvent; and an auxiliary solvent different from the main solvent and present in an amount of less than 50 wt % in the good solvent. Preferably, the weight ratio of the main solvent of the good solvent and the auxiliary solvent of the good solvent may be 50:50 to 99:1.

In an exemplary embodiment of the present specification, the main solvent of the good solvent basically means a solvent with a ratio of 50% or more in the entire good solvent, and has an advantage in that it is possible to include both a solvent in which interaction with a polymer is further considered when the polymer is dissolved and a solvent having a property of swelling the polymer well by mixing two or more solvents.

In an exemplary embodiment of the present specification, the good solvent in which the main solvent and the auxiliary solvent are mixed has a PVC solubility score value of 11 or more according to Equation 1 in consideration of the mixing ratio of each solvent in the mixed solvent, that is, the weight fraction, and is selected in consideration of the difference in solubility between the non-solvent and polyvinyl chloride used together.

In an exemplary embodiment of the present specification, the main solvent of the good solvent may be selected among n,n-dimethylacetamide, cyclopentanone, 2-pentanone, dioxane, nitrobenzene, ethylacetate, benzene, trichloroethylene, 1,2-dichloroethane, chloroform, cyclohexanone, tetrahydrofuran, 2-butanone, acetone, and 4-methylpentan-2-one.

In an exemplary embodiment of the present specification, the auxiliary solvent of the good solvent may be selected among n,n-dimethylacetamide, cyclopentanone, 2-pentanone, dioxane, nitrobenzene, ethylacetate, benzene, trichloroethylene, 1,2-dichloroethane, chloroform, cyclohexanone, tetrahydrofuran, 2-butanone, acetone, 4-methylpentan-2-one, 1,2,3-propanetrioltrinitrate, tetramethylenesulfoxide, 1-piperidinecarboxaldehyde, 1-acetylpiperidine, n-methyl-2-pyrrolidinone, 1,4-dimethylpyrrolidone, 1,3-dimethyl-2-imidazolidinone, 1,5-dimethylpyrrolidone, hexamethylphosphoramide, dimethylpropyleneurea, 1-ethyl-2-pyrrolidone, 1,3-dimethylpyrrolidone, 4-acetylmorpholine, 1-[ethoxy(methyl)phosphoryl]methylpiperidine, n,n-dimethylpropanamide, tributylphosphineoxide, 1-propyl-2-pyrrolidinone, n-isopropylpyrrolidone, n,n,4-trimethylbenzamide, n-methyl-o-acetotoluidide, 2-cyclohexen-1-one, n,n,3-trimethylbenzamide, n,n-dimethylbenzamide, trimethylenesulfide, n,n,2-trimethylbenzamide, 1,3-dioxane, tetramethylurea, 2-azacyclooctanone, 4-methyl-1-propyl-2-pyrrolidinone, 5-methyl-1-propyl-2-pyrrolidinone, n,n-diethylacetamide, 1,3-propanedithiol, 4,5-dihydro-2-methylthiazole, pyrrolidine, 3-methoxy-n,n-dimethylpropanamide, 4-fluoro-n,n-dimethylbenzamide, cycloheptanone, 1-morpholinopentan-1-one, 3-methylcyclohexanone, n,n-dimethylbutanamide, caprolactam, tetrahydrothiophene, 2-methoxy-n,n-dimethylbenzamide, dimethylformamide, 1,2-ethanedithiol, 1,4,7,10-tetraoxacyclododecane, ethyl 1,3-dimethyl-2-oxopyrrolidine-3-carboxylate, 1-(4-methylpiperidyl)butan-1-one, 2-methylcyclohexanone, 1-(2,6-dimethylmorpholin-4-yl)butan-1-one, n,n-diethylformamide, 4-(propan-2-yl)phenyldimethylcarbamate, n,n-dimethyl-3-phenylpropanamide, n-(1-methyl-2-methoxyethyl)-n-chloroacetyl-2-ethyl-6-methylaniline, dichloroacetylchloride, n,n-diethyl-3-(2-methyl-1,3-dioxolan-2-yl)propanamide, n-acetylcaprolactam, d-camphor, n-methylformanilide, ethyl n-acetyl-n-ethyl-beta-alaninate, (2,5-dimethoxyphenyl)acetaldehyde, 4-methylcyclohexanone, 2,6-dimethyl-2,5-cyclohexadiene-1,4-dione, 4-vinylcyclohexene dioxide, nicotine, 2-methylcyclopentanone, dimethylveronal, diethyl(trichloromethyl)phosphonate, 3-methyl-6-isopropylidenecyclohexanone, 1-heptanoyl-4-methylpiperazine, n,n-diethyl-4-oxopentanamide, n-ethyl-n-phenylacetamide, dimethyldisulfide, 4,6-dimethylpyrimidine, 1,4-oxathiane, n-sec-butylpyrrolidone, 1-(4-methylpiperidin-1-yl)pentan-1-one, 1-butyl-5-methyl-2-pyrrolidinone, 3-methyl-2-oxazolidone, n,n-dimethyl-4-(propan-2-yl)benzamide, 2,5-dimethylpyrazine, mesitenelactone, propyl 4-(dimethylamino)-4-oxobutanoate, diethyldimethylamidophosphate, isophorone, n,n-dimethyl 4-methoxybenzamide, 1,2,3,5-tetramethoxybenzene, 3,4,5-trimethoxytoluene, caprolactone, 3-methyl-piperidin-2-one, 2-ethylhexylsalicylate, ethyl 3-ethyl-2-oxooxolane-3-carboxylate, 1-ethyl-3,5,7-trimethyl-1,3-dihydro-azepin-2-one, dimethylisosorbide, cyclotenebutyrate, ethyl 2-(2-methoxyphenoxy)acetate, cyrene, 2,4,4-trimethyl-6-oxocyclohex-1-en-1-yl acetate, 1,2-benzodihydropyrone, tripropyleneglycolethyletherisomer, 3,4-dimethoxypropiophenone, methylaminoacetaldehydedimethylacetal, methyl 2,6-dimethoxybenzoate, 1,3-dimethyl-1,2,3,4-tetrahydro-4-quinolinone, ethyl 3,5-dimethoxybenzoate, 4-methoxybenzaldehyde, ethyl (4-methylphenoxy)acetate, 2,3,4-trimethoxyacetophenone, ethylimidazole, methyl 2-oxocyclooctanecarboxylate, ethyl 2-methyl-4-oxocyclohex-2-enecarboxylate, 2′,5′-dimethoxyacetophenone, isobutylformate, ethyl 2-(2,6-dimethylphenoxy)acetate, ethyl 2-(benzyloxy)acetate, hexylacetoacetate, diethylglutarate, 4-carbethoxy-2-ethyl-3-methyl-2-cyclohexen-1-one, nepetalactone, tripropyleneglycolbutylether, ethyl 5,5-dimethyl-2-oxofuran-3-carboxylate, 2-ethoxybenzaldehyde, methyl-2,4-dimethoxybenzoate, 5,5-diethyl-4-methoxyfuran-2(5h)-one, ethyl 4,6-dimethyl-2-oxo-3,4-dihydro-2h-pyran-5-carboxylate, ethyl 4-phenoxybutanoate, 2,5-dimethoxypropiophenone, ethyl 5,5-dimethyl-2-oxotetrahydrofuran-3-carboxylate, ethyl 3-(5-oxotetrahydrofuran-2-yl)propanoate, benzylsalicylate, n,n-diethyl-4-methyl-4h-pyridine-1-carboxamide, p-tolylacetonitrile, 5-methyl-2(3h)-furanone, 1-methyl-2-(3-pyridyl)pyrrole, formicacidpentylester, ethyl 4-(dimethylamino)benzoate, n-butylacetamide, 4-methylbenzaldehyde, 3′,4′-dimethoxyisobutyrophenone, benzaldehyde, 1,2,4-triethoxybenzene, isobutylphenoxyacetate, m-dimethoxybenzene, 3-oxobutanoicacid-1-methylethylester, ethyl2-(cyclohexylmethyl)-3-oxobutanoate, 5-ethyldihydro-2 (3h)-furanone, 2-isopropyl-n,2,3-trimethylbutyramide, 4-(4-methylpiperidino)pyridine, 3-oxaspiro-undecan-2-one, formicacidhexylester, methyl 2,2-dimethyl-4,6-dioxocyclohexanecarboxylate, ethyl 6-ethyl-2-methyl-4-oxocyclohex-2-ene-1-carboxylate, benzenepropanenitrile, 4,4-dimethyl-5,6,7,8-tetrahydro-1H-isochromen-3-one, ethyl 2,6-dimethyl-4-oxocyclohex-2-enecarboxylate, n,n-dimethyloctanamide, 1-(2,5-dimethylpyrrolidin-1-yl)pentan-1-one, gamma-butyrolactone, t-butylacetoacetate, 4-(methoxymethyl)anisole, mintlactone, 2-methoxyethylacetoacetate, p-(2,2-dimethoxyethoxy)anisole, 1-(1-ethylpropyl)-2-pyrrolizinone, 2,5-dimethoxytetrahydrofuran, 2-acetoxy-1-ethoxypropane, 1,3-dimethyl-4-ethylcarbostyril, 1-(4-methoxyphenoxy)acetone, glycerol tripropionate, 3-dimethylaminoanisole, 2,4-dimethylbenzaldehyde, paraldehyde, glyceryl tributyrate, 2,3,4,5-tetramethoxytoluene, 1,3,3-trimethyl-2h-quinolin-4-one, 3-cyclohexene-1-carboxaldehyde, isobutylsalicylate, 2-(1-methylpropyl)phenyl methylcarbamate, (2,2-diethoxyethoxy)benzene, methyl 3-(5-oxooxolan-2-yl)propanoate, 4-acetoxy-2,5-dimethyl-3(2h)-furanone, 3-ethylchroman-2-one, ethyl 3-cyclohexyl-3-oxopropanoate, 1-propyl-2(1h)-pyridinone, 7-methyl-4,4a,5,6-tetrahydro-3H-naphthalen-2-one, 1-(3,5-dimethylphenoxy)propan-2-one, ethyl 3-(2,5,5-trimethyl-1,3-dioxan-2-yl)propanoate, 1-tetralone, 4-methyl-3-penten-2-one, 1-tert-butylpyrrolidin-2-one, methyl 2-(4-(tert-butyl)phenoxy)acetate, 2,6,10,10-tetramethyl-1-oxaspiro(4,5)deca-2,6-dien-8-one, ethyl 4-ethoxybenzoate, maltyl isobutyrate, 4-methyl-1,3-dioxane, 2-acetylpyridine, 1,4-diethoxybenzene, 1-(2,2-diethoxyethoxy)-4-methylbenzene, cyclohexyl lactone, 2-(2-butoxyethoxy)ethyl acetate, n-methylbenzamide, 3-acetyl-5-propyldihydrofuran-2 (3h)-one, 2,2-diethoxyethoxymethylbenzene, n-methyl-m-tolylcarbamate, ethylene glycol dibutyrate, 4-cyano-2-fluorobenzoyl chloride, 3,6-dimethyl-2,3,3a,4,5,7a-hexahydro-1-benzofuran-2-one, ethyl 2-(2-oxocyclohexyl)acetate, 4-(2-acetoxyethoxy)toluene, 4-(2,5-dimethylpyrrol-1-yl)-2,6-dimethylmorpholine, 1-butylpyridin-2(1h)-one, n,n-dimethyldecanamide, valeraldehyde, 2-(3-pyridyl)piperidine, 4-methoxypropiophenone, 4-tert-butyl-n,n-dimethylbenzamide, acetylacetone, ethyl 2-methyl-2-phenoxypropanoate, ethyl 2-acetyl-5-oxohexanoate, n-sec-isopentylpyrrolidone, 4-methyl-1-tetralone, 2-butoxyethanolacetate, isobutyric anhydride, n-hexyl-2-pyrrolidinone, 3-benzyl-2,4-pentanedione, ethyl 5-oxodecanoate, methyl 2-hexyl-3-oxocyclopentanecarboxylate, 8-ethyl-1-oxaspiro[4.5]decan-2-one, n,n′-dibutylethyleneurea, methyl 3-oxododecanoate, 3-methyl-5-isopropyl-4-carbethoxy-2-cyclohexene-1-one, 6-pentyl-2h-pyran-2-one, m-methoxyacetophenone, benzisoxazole, n,n-diethylnicotinamide, ethyl 3-(2-oxocyclohexyl)propanoate, propyl 4-ethoxybenzoate, 5-butyl-5-ethyltetrahydro-2h-pyran-2-one, ethyl 2-oxo-1-propylcyclohexanecarboxylate, 2-propoxyethylacetate, dimethyl 2-methylglutarate, o-methylcorypalline, 1-pentylimidazole, n-formylmorpholine, methyl 2-butoxybenzoate, 7-butyloxepan-2-one, propanoic anhydride, acetophenone, 1-(3-methylpiperidin-1-yl)pentan-1-one, 1-butyl-2-methyl-imidazole, anisaldehyd diethyl acetal, 1-(2-methylpiperidin-1-yl)pentan-1-one, 5-butyl-5-ethyloxolan-2-one, 2,4,6-trimethylbenzaldehyde, n,n-diethylcyclohex-3-ene-1-carboxamide, butan-2-yl 4-methoxybenzoate, propylene glycol alpha methylbenzyl ether, 5-(benzyloxy)pentan-2-one, n,n-dimethyldodecanamide, n,n-diethyl-m-toluamide, (s)-dihydrobovolide, 3,4-dihydro-3-oxoedulan, butyl 2-methoxybenzoate, 7-cyclohexyloxepan-2-one, dimethyl 1,4-cyclohexanedicarboxylate, delta-decalactone, ethyl 2,5-dimethylphenoxyisobutyrate, 4a,8-dimethyl-3,4,5,6,7,8-hexahydronaphthalen-2-one, 5-hydroxy-2-decenoic acid lactone, ethyl 2-(2-oxooxolan-3-yl)acetate, methyloctalacton, 3-pentanone, 2,4-diacetylfuran, 2′-pentyloxyacetophenone, furfural, diethylmalonate, 1-(azepan-1-yl)butan-1-one, p-methoxyvalerophenone, n,n-diethylbenzamide, 4,4-dimethylnaphthalen-1(4h)-one, gamma-decalactone, piperitone, ethylacetoacetate, 1-hexylimidazole, dihydromintlactone, 4-heptanone, dimethyl glutarate, 4-(benzyloxy)-4-methylpentan-2-one, 3,3-dimethyl-1-phenoxybutan-2-one, molinate, 7-methoxy-2,2-dimethylchromene, propiophenone, ethyl 2-benzoyl-3-methylbutanoate, n,n-diethyl-2-methylcyclohexane-1-carboxamide, 5-methyl-5-propyl-1,3-dioxan-2-one, 3-methyl-5-(2-oxopropyl)oxolan-2-one, 1-(azepan-1-yl)pentan-1-one, 2-(2-ethoxyethoxy)-ethanolacetate, 2-methyl-1-(1-phenylethyl)imidazole, methyl 6-(diethylamino)-6-oxohexanoate, 5-undecanolide, 4-methyl-5-pentyloxolan-2-one, 2-ethoxyethylbenzoate, 3,4-dihydro-2(1h)-naphthalenone, 1-methoxy-3-(4-methylphenoxy)benzene, n,n-dihexylacetamide, dihydroactinidiolide, 1-pentanoylpiperidine, propan-2-yl 6-(dimethylamino)-6-oxohexanoate, 5,5-dimethyl-4-(3-oxobutyl)oxolan-2-one, 1-octylpyrrolidin-2-one, gamma-undecalactone, 4-ethoxyphenethylacetate, 2-hexanoylcyclohexan-1-one, gamma-methyldecalactone, 1-(azepan-1-yl)-3-methylbutan-1-one, methyl dihydrojasmonate, p-methoxypivalophenone, 2-acetyl-4-tert-butylcyclohexan-1-one, 4,4-dibutyl-gamma-butyrolactone, 4-ethyl-2,6-dimethoxyphenol, n,n-diethylcyclohexanecarboxamide, 5-dodecanolide, 1-hexanoylpiperidine, 1-heptyl-1h-imidazole, 2-isopropoxyethylbenzoate, butanoic anhydride, methylacetoacetate, 2,3,3-trimethylindanone, 5-hexyl-4-methyldihydrofuran-2 (3h)-one, phenoxyacetone, 1-propoxy-2-propanol, ethyl di-n-propylthiolcarbamate, 1,2-dimethoxy-4-methylbenzene, tetrahydrofurfurylpropionate, 2-pyrrolidon, 2-methoxypropyl acetate, 4-dodecanolide, 2-ethoxy-4-(methoxymethyl)phenol, n-isopentylpropionanilide, pentylpiperidine-1-carboxylate, 1-(2-ethylpiperidin-1-yl)butan-1-one, isopropyl 2-isopropoxy-benzoate, n,n-dibutylpentanamide, 2,3-dihydro-2,2-dimethyl-7-hydroxybenzofuran, 2-heptanone, 2-[2-(2-methoxypropoxy)propoxy]-1-propanol, 1,2,3,4,5,6,7,8-octahydro-2,8,8-trimethylnaphthalene-2-carbaldehyde, cycloate, 4-methylquinoline, 5-nonanone, n,n-dipentylbutanamide, 3-dimethylamino-2-methylpropiophenone, 2-ethyl-1-pentanoylpiperidine, 4-(ethoxymethyl)-2-methoxyphenol, n,n-dibutylhexanamide, 2-ethylhexyllactate, tripropyleneglycolmonomethylether, magnolione, 1-octylimidazole, butylformate, butylate, 5-ethyl-2-(2-furyl)-4-propyl-m-dioxane, 1-methoxy-4-(2-methoxyethoxymethyl)benzene, hexylpiperidine-1-carboxylate, 4-propylcyclohexanone, 2,5,8,11,14,17-hexaoxaoctadecane, ethyl n,n-diethylsuccinamate, ethyl dipentylcarbamate, 2-butoxyethyl 4-methylbenzoate, 4′-pentylacetophenone, vernolate, 2-cyclopentyl cyclopentanone, 2-butoxyethylbenzoate, trimethoxymethane, phenoyethylpropionate, 4′-isobutylacetophenone, 2-methylindole, n,n,2-triethylhexanamide, 2-methoxy-4-propylphenol, 3,5-bis(2-methylpropyl)cyclohexane-1,2-dione, 2,4-diethoxy-6-methylpyrimidine, ethyl-2-hydroxybenzoate, p-ethoxybenzaldehyde, glycerol triacetate, p-hexylacetophenone, n,n-diisopropyl-phenylacetamide, 3-ethylundecane-4,6-dione, 1-benzazine, methyl 5-methylsalicylate, n,n-dipropylheptanamide, 4-methylpyridine, 4-pentylcyclohexanone, 4-pyridinecarboxaldehyde, oxolan-2-yl methylheptanoate, 2-(2-(4-methylcyclohex-3-enyl)propyl)cyclopentanone, 2-isopropoxyphenol, octanophenon, 2-benzyl-4-methyl-1,3-dioxane, ethylbenzoate, 4-methyl-2-(2-methylpropyl)oxan-4-ol, 3-(diethylamino)propiophenone, benzylacetoacetate, isoamylbenzoate, pyridine, dichloromethane, or 1,1,2,2-tetrachloroethane, but is not limited thereto.

In an exemplary embodiment of the present specification, the non-solvent is any one of an alcohol-based solvent, a ketone-based solvent, an ester-based solvent, an ether-based solvent, a polar nonprotic solvent, a hydrocarbon solvent, or a halogen-containing solvent.

In an exemplary embodiment of the present specification, the non-solvent may include a main solvent present in an amount of 50 wt % or more in the non-solvent; and an auxiliary solvent different from the main solvent and present in an amount of less than 50 wt % in the non-solvent. Preferably, the weight ratio of the main solvent of the non-solvent and the auxiliary solvent of the non-solvent may be 50:50 to 99:1.

In an exemplary embodiment of the present specification, the main solvent of the non-solvent basically means a solvent with a ratio of 50% or more in the entire non-solvent, and has an advantage in that it is possible to include both a solvent in which interaction with a polymer is further considered when the polymer is dissolved and a solvent having a property of swelling well the polymer by mixing two or more solvents.

In an exemplary embodiment of the present specification, the single non-solvent has a PVC solubility score value of less than 11 according to Equation 1, and is selected in consideration of the difference in solubility between the good solvent and polyvinyl chloride used together.

In an exemplary embodiment of the present specification, the single non-solvent may be selected among propanol, diethylether, n-heptane, hexane, 1,4-butadiol, octane, cyclopentane, propyleneglycol, dimethylsulfoxide, methanol, ethanol, 2-propanol, 2,4-dimethylhexane, 3-ethyl-2,4-dimethylpentane, 2,7-dimethyloctane, 2,6-dimethyl-heptane, 2-methyl-3-ethylpentane, 3,3-dimethylhexane, 2,3,5-trimethylhexane, 2,3,4-trimethylpentane, 2,2-dimethylheptane, 3,3-dimethylpentane, 1,1,1,2,2,3,3,4,4-nonafluorotetracosane, 2,5-dimethylhexane, 2,2-dimethylhexane, 2,3,3-trimethylpentane, formicacid, 2,2,6-trimethylheptane, 2,2,4-trimethylhexane, 2,2,3-trimethylpentane, 2,3,3,4-tetramethylpentane, 2,4,4-trimethylhexane, 1-hydroxybenzotriazole, 3-ethyl-2,2-dimethyl-pentane, 2,2,4,6,6-pentamethylheptane, 2,2,5-trimethylhexane, 2-nitroethanol, 2,2,3,4-tetramethylpentane, octamethyltrisiloxane, 2,2,3,3-tetramethylhexane, 1,5-pentanediol, 1,1,1,2,2,3,3-heptafluoroundecane, 2,2,5,5-tetramethylhexane, 2,2,3,3-tetramethylpentane, 2,2,4-trimethylpentane, 2,2,4,4-tetramethylpentane, dodecamethylcyclohexasiloxane, acetaldehydeammonia, 1,1,1,2,2,3,3,4,4-nonafluorohexadecane, dodecamethylpentasiloxane, 1,1,1,2,2,3,3-heptafluorononane, 1,3-dioxolan-2-one, decamethyltetrasiloxane, 1-(perfluorohexyl)docosane, hexamethyldisilane, 1,1,1,2,2,3,3,4,4,5,5-undecafluorononadecane, tetradecamethylhexasiloxane, 1,1,1,2,2,3,3,4,4,5,5,6,6-tridecafluorohexacosane, 1,1,1,2,2,3,3,4,4-nonafluoroundecane, 1,1,1,2,2,3,3,4,4-nonafluorododecane, hexadecamethylheptasiloxane, 1,1,1,2,2,3,3,4,4,5,5,6,6-tridecafluorotetracosane, propynol, 1,1,1,2,2,3,3,4,4-nonafluorodecane, ethanolamine, 1,1,1,2,2,3,3,4,4,5,5,6,6-tridecafluorodocosane, 2-hydroxypropanenitrile, 1,1,1,2,2,3,3,4,4,5,5,6,6-tridecafluoroicosane, 1,1,1,2,2,3,3,4,4-nonafluorononane, 1,1,1,2,2,3,3,4,4,5,5,6,6-tridecafluorooctadecane, cis-2-butene-1,4-diol, 1,1,1,2,2,3,3,4,4,5-decafluorononane, 1,1,1,2,2,3,3,4,4,5,5,6,6,7,7,8,8-heptadecafluorooctacosane, 1,1,1,2,2,3,3,4,4,5,5,6,6-tridecafluorohexadecane, 1,1,1,2,2,3,3,4,4,5,5,6,6,7,7,8,8-heptadecafluorohexacosane, 1,1,1,2,2,3,3,4,4,5,5,6,6,7,7,8,8-heptadecafluoropentacosane, 1,1,1,2,2,3,3,4,4,5,5,6,6-tridecafluoropentadecane, 1,1,1,2,2,3,3,4,4,5,5,6,6,7,7,8,8-heptadecafluorotetracosane, 1-(perfluorohexyl)octane, 1,1,1,2,2,3,3,4,4,5,5-undecafluorodecane, 1,1,1,2,2,3,3,4,4,5,5,6,6-tridecafluorotridecane, 1,1,1,2,2,3,3,4,4,5,5,6,6,7,7,8,8-heptadecafluorodocosane, 1h,8h-hexadecafluorooctane, 1,1,1,2,2,3,3,4,4,5,5,6,6,7,7,8,8,9,9,10,10-henicosafluorotriacontane, hexacosamethyldodecasiloxane, 1,1,1,2,2,3,3,4,4,5,5,6,6,7,7,8,8-heptadecafluoroicosane, 1,1,1,2,2,3,3,4,4,5,5,6,6-tridecafluorododecane, 1,3-propanediol, 1,1,1,2,2,3,3,4,4,5,5,6,6-tridecafluoroundecane, 1,1,1,2,2,3,3,4,4,5,5,6,6,7,7,8,8-heptadecafluorononadecane, 1,1,1,2,2,3,3,4,4,5,5,6,6,7,7,8,8,9,9,10,10-henicosafluorononacosane, 1,1,1,2,2,3,3,4,4,5,5,6,6,7,7,8,8-heptadecafluorooctadecane, 1,1,1,2,2,3,3,4,4,5,5,6,6,7,7-pentadecafluorotetradecane, 1,1,1,2,2,3,3,4,4,5,5,6-dodecafluorononane, 1,1,1,2,2,3,3,4,4,5,5-undecafluorooctane, 1,1,1,2,2,3,3,4,4,5,5,6,6,7,7-pentadecafluorotridecane, 1-perfluorooctyloctane, 1,1,1,2,2,3,3,4,4,5,5,6,6-tridecafluorodecane, 1,1,1,2,2,3,3,4,4,13,13,14,14,15,15,16,16,16-octadecafluorohexadecane, 1,1,1,2,2,3,3,4,4,5,5,6,6,7,7,8,8-heptadecafluoropentadecane, 1,1,1,2,2,3,3,4,4,5,5,6,6,7,7,8,8,9,9,10,10,11,11,12,12-pentacosafluorohentriacontane, formicin, 1,1,1,2,2,3,3,4,4,5,5,6,6,7,7,8,8-heptadecafluorotetradecane, 1-(henicosafluorodecyl)undecane, 1,1,1,2,2,3,3,4,4,5,5,6,6,7,7,8,8-heptadecafluorotridecane, 1,1,1,2,2,3,3,4,4,5,5,6,6,7,7,8,8,9,9,10,10,11,11,12,12-pentacosafluorooctacosane, ethylenecyanohydrin, 1,1,1,2,2,3,3,4,4,5,5,6,6,7,7,8,8,9,9,10,10-henicosafluoroicosane, 1,1,1,2,2,3,3,4,4,5,5,6,6-tridecafluorooctane, perfluorooctylbromide, 1,1,1,2,2,3,3,4,4,5,5,6,6,7,7,8,8,9,9,10,10-henicosafluorooctadecane, 1,1,1,2,2,3,3,4,4,5,5,6,6,7,7,8,8-heptadecafluorododecane, 1,1,1,2,2,3,3,4,4,5,5,6,6,7,7,8,8,9,9,10,10,11,11,12,12-pentacosafluorotetracosane, pentacosafluorotricosane, 1-(perfluorohexyl)propane, 1,1,1,2,2,3,3,4,4,5,5,6,6,7,7,8,8,9,9,10,10-henicosafluoropentadecane, 1,1,1,2,2,3,3,4,4,5,5,6,6,7,7,8,8-heptadecafluorodecane, 1,1,1,2,2,3,3,4,4,5,5,6,6,7,7,8,8,9,9,10,10-henicosafluorotetradecane, 1,1,1,2,2,3,3,4,4,5,5,6,6,7,7,8,8,9,9,10,10,11,11,12,12-pentacosafluorooctadecane, 1,1,1,2,2,3,3,4,4,5,5,6,6,7,7,8,8,9,9,10,10-henicosafluorotridecane, 1,1,1,2,2,3,3,4,4,5,5,6,6,7,7,8,8,9,9,10,10-henicosafluorododecane, glycol, tetratriacontafluorotetracosane, glycerol, 1,1,1,2,2,3,3,4,4,5,5,6,6,7,7,8,8,9,9,10,10,11,11,12,12-pentacosafluorotetradecane, methylhydroperoxide, H₂O, 4-hydroxymethyl-1,3-dioxolan-2-one, hydroxyacetonitrile, perfluorooctane, cis-perfluorodecalin, methanesulfonicacid, formamide, 4-methylphenol, PBr₃, H₂O₂, BBr₃, methylbutane, 2,2-dimethylbutane, 2,2,3-trimethyl-butane, 2,2-dimethylpentane, 2,3-dimethylbutane, pentane, 2,4-dimethylpentane, 2-methylpentane, 2-methylhexane, 3-methylpentane, 3-methyl-hexane, 3-ethylpentane, 3-ethyl-3-methylpentane, 2-methylheptane, 3,3-diethylpentane, 3-methylheptane, 2-methyloctane, 4-methylheptane, 3-ethylheptane, 2-methyl-nonane, 3-methyloctane, 4-methyloctane, 2-methyldecane, 3-methyl-nonane, 1,1,3,3-tetramethylcyclopentane, 1,3-butanediol, 4-methyldecane, 5-methyl-nonane, 4-methyl-nonane, 1,1-dimethylcyclopentane, 1,1,2-trimethylcyclopentane, 1,1,2-trimethylcyclohexane, n-decane, n-nonane, isobutylcyclopentane, (1-methylethyl)-cyclopentane, tert-butylcyclohexane, pentadecane, tridecane, 1,1,3-trimethylcyclopentane, 1,1,3-trimethylcyclohexane, nonadecane, heptadecane, octadecane, ethylcyclopentane, dodecane, n-undecane, propylcyclopentane, isobutylcyclohexane, isopropylcyclohexane, 1-ethyl-1-methylcyclopentane, 2,3,3-trimethyl-1-butene, 2,4,4-trimethyl-1-pentene, n-hexylcyclopentane, pentylcyclopentane, 1-isopropyl-4-methylcyclohexane, trimethylaluminum, n-tetradecylcyclopentane, tridecylcyclopentane, tetradecane, n-dodecylcyclopentane, n-decylcyclopentane, n-propylcyclohexane, ethylcyclohexane, 3,3-dimethyl-1-butene, n-hexadecane, pentene, 2,3,3-trimethyl-1-pentene, decylcyclohexane, pentylcyclohexane, 3,4-dicyclohexyl-2,5-dimethylhexane, 2-methyl-1-butene, 3,3-dimethyl-1-pentene, 4,4-dimethyl-1-pentene, butylcyclohexane, methylcyclopentane, methylcyclohexane, decahydronaphthalene, hexahydroindene, bicyclohexyl, cyclohexane, 2,4,4-trimethyl-2-pentene, 2,3-dimethyl-1-pentene, tricyclohexylmethane, butylcyclopentane, 1-hexene, 2,3-dimethyl-1-butene, methylformamide, 3,3-dimethyl-1-butyne, triethyleneglycol, 3-methoxy-1,2-propanediol, 2,4-dimethyl-1-pentene, 1-heptene, 3-methyl-1-pentene, 3-ethyl-2-methyl-1-pentene, 1,4-bis(1,1-dimethylethyl)-benzene, diethyleneglycol, octane, methoxyaceticacid, 1-nonene, 4-methyl-1-hexene, 1,2,3,4-tetrahydro-1,1,3,4,4,6-hexamethylnaphthalene, 1,3,5-triisopropylbenzene, 1,2,2,6,6-pentamethylpiperidine, 1,4-diethylbenzene, 2-methyl-2-butene, isoprene, 4-methyl-1-pentene, 1-tetradecene, 3-ethyl-1-pentene, 2-methyl-3-heptene, 1-decene, 2,3,4-trimethyl-2-pentene, 1-nonadecene, butylbenzene, 2-ethoxy-2-methyl-propane, 1,2-diethylbenzene, ethanedial, cyclohexylbenzene, galaxolide, 1,2,3,4-tetramethylbenzene, 1,2,3,5-tetramethylbenzene, pentaethylbenzene, 1,3,5-trimethylbenzene, isobutanol, peroxyaceticacid, 1,6-hexanediol, 2,6-di-tert-butyl-4-methylpyridine, 2,2′-(methylimino)bis-ethanol, 1,3-bis(1-methylethyl)-benzene, 2,7-diisopropylnaphthalene, 1,2,3,5-tetraethylbenzene, tetrahydro-2,2,5,5-tetramethylfuran, 1,5-diisopropylnaphthalene, 2,3-butanediol, 1,2-diisopropylbenzene, benzene, 1-pentan-2-yl-4-pentan-3-ylbenzene, tetradecylbenzene, pentadecylbenzene, ethylbenzene, 3,6-bis-(methoxymethyl)-durene, himachalen_gamma, 1-(1,1-dimethylethyl)-3-methylbenzene, tridecylbenzene, 1-(methoxymethyl)-4-(2,4,4-trimethylpentan-2-yl)benzene, dodecylbenzene, 1,2,4-trimethylbenzene, 1-cyclohexyl-2-isopropylbenzene, dl-pin-2(3)-ene, 2,6-di-tert-butylpyridine, 1,1′-oxybis-octane, 1,5-hexanediol, 1-(1,1-dimethylethyl)-3,5-dimethylbenzene, menthylisovalerate, 1-methyl-7-(1-methylpropyl)naphthalene, tert-butanol, himachalen_beta, dimethoxymethane, 1,2,3,4,5,6,7,8-octahydroanthracene, (1,1-dimethylethyl)benzene, 1,2-hexanediol, cyclohexene, perhydrofarnesylacetone, delta-cadinene, isopropylbenzene, undecylbenzene, 2,5-hexanediol, dipropyleneglycol, 1,6-dimethyl-4-isopropyltetralin, di-n-propylether, diisopropylether, cuparen, 1,4-bis(1-methylethyl)-benzene, 2,4-dimethyl-3-thiapentane, 1-methoxy-4-(2,4,4-trimethylpentan-2-yl)benzene, 1-ethoxybutane, decylbenzene, orcycloheptane, but is not limited thereto.

In an exemplary embodiment of the present specification, the non-solvent in which the main solvent and the auxiliary solvent are mixed has a PVC solubility score value of less than 11 according to Equation 1 in consideration of the mixing ratio of each solvent in the mixed solvent, that is, the weight fraction, and is selected in consideration of the difference in solubility between the good solvent and polyvinyl chloride used together.

In an exemplary embodiment of the present specification, the main solvent of the non-solvent may be selected among propanol, diethylether, n-heptane, hexane, 1,4-butadiol, octane, cyclopentane, propyleneglycol, dimethylsulfoxide, methanol, ethanol, or 2-propanol.

In an exemplary embodiment of the present specification, the auxiliary solvent of the non-solvent may be selected among propanol, diethylether, n-heptane, hexane, 1,4-butadiol, octane, cyclopentane, propyleneglycol, dimethylsulfoxide, methanol, ethanol, 2-propanol, 2,4-dimethylhexane, 3-ethyl-2,4-dimethylpentane, 2,7-dimethyloctane, 2,6-dimethyl-heptane, 2-methyl-3-ethylpentane, 3,3-dimethylhexane, 2,3,5-trimethylhexane, 2,3,4-trimethylpentane, 2,2-dimethylheptane, 3,3-dimethylpentane, 1-(perfluorobutyl)icosane, 2,5-dimethylhexane, 2,2-dimethylhexane, 2,3,3-trimethylpentane, formicacid, 2,2,6-trimethylheptane, 2,2,4-trimethylhexane, 2,2,3-trimethylpentane, 2,3,3,4-tetramethylpentane, 2,4,4-trimethylhexane, 1-hydroxybenzotriazole, 3-ethyl-2,2-dimethyl-pentane, 2,2,4,6,6-pentamethylheptane, 2,2,5-trimethylhexane, 2-nitroethanol, 2,2,3,4-tetramethylpentane, octamethyltrisiloxane, 2,2,3,3-tetramethylhexane, 1,5-pentanediol, 1,1,1,2,2,3,3-heptafluoroundecane, 2,2,5,5-tetramethylhexane, 2,2,3,3-tetramethylpentane, 2,2,4-trimethylpentane, 2,2,4,4-tetramethylpentane, dodecamethylcyclohexasiloxane, acetaldehydeammonia, 1,1,1,2,2,3,3,4,4-nonafluorohexadecane, dodecamethylpentasiloxane, 1,1,1,2,2,3,3-heptafluorononane, 1,3-dioxolan-2-one, decamethyltetrasiloxane, 1-(perfluorohexyl)docosane, hexamethyldisilane, 1,1,1,2,2,3,3,4,4,5,5-undecafluorononadecane, tetradecamethylhexasiloxane, 1,1,1,2,2,3,3,4,4,5,5,6,6-tridecafluorohexacosane, 1,1,1,2,2,3,3,4,4-nonafluoroundecane, 1,1,1,2,2,3,3,4,4-nonafluorododecane, hexadecamethylheptasiloxane, 1,1,1,2,2,3,3,4,4,5,5,6,6-tridecafluorotetracosane, propynol, 1,1,1,2,2,3,3,4,4-nonafluorodecane, ethanolamine, 1,1,1,2,2,3,3,4,4,5,5,6,6-tridecafluorodocosane, 2-hydroxypropanenitrile, 1,1,1,2,2,3,3,4,4,5,5,6,6-tridecafluoroicosane, 1,1,1,2,2,3,3,4,4-nonafluorononane, 1,1,1,2,2,3,3,4,4,5,5,6,6-tridecafluorooctadecane, cis-2-butene-1,4-diol, 1,1,1,2,2,3,3,4,4,5-decafluorononane, 1,1,1,2,2,3,3,4,4,5,5,6,6,7,7,8,8-heptadecafluorooctacosane, 1,1,1,2,2,3,3,4,4,5,5,6,6-tridecafluorohexadecane, 1,1,1,2,2,3,3,4,4,5,5,6,6,7,7,8,8-heptadecafluorohexacosane, 1,1,1,2,2,3,3,4,4,5,5,6,6,7,7,8,8-heptadecafluoropentacosane, 1,1,1,2,2,3,3,4,4,5,5,6,6-tridecafluoropentadecane, 1,1,1,2,2,3,3,4,4,5,5,6,6,7,7,8,8-heptadecafluorotetracosane, 1-(perfluorohexyl)octane, 1,1,1,2,2,3,3,4,4,5,5-undecafluorodecane, 1,1,1,2,2,3,3,4,4,5,5,6,6-tridecafluorotridecane, 1,1,1,2,2,3,3,4,4,5,5,6,6,7,7,8,8-heptadecafluorodocosane, 1h,8h-hexadecafluorooctane, 1,1,1,2,2,3,3,4,4,5,5,6,6,7,7,8,8,9,9,10,10-henicosafluorotriacontane, hexacosamethyldodecasiloxane, 1,1,1,2,2,3,3,4,4,5,5,6,6,7,7,8,8-heptadecafluoroicosane, 1,1,1,2,2,3,3,4,4,5,5,6,6-tridecafluorododecane, 1,3-propanediol, 1,1,1,2,2,3,3,4,4,5,5,6,6-tridecafluoroundecane, 1,1,1,2,2,3,3,4,4,5,5,6,6,7,7,8,8-heptadecafluorononadecane, 1,1,1,2,2,3,3,4,4,5,5,6,6,7,7,8,8,9,9,10,10-henicosafluorononacosane, 1,1,1,2,2,3,3,4,4,5,5,6,6,7,7,8,8-heptadecafluorooctadecane, 1,1,1,2,2,3,3,4,4,5,5,6,6,7,7-pentadecafluorotetradecane, 1,1,1,2,2,3,3,4,4,5,5,6-dodecafluorononane, 1,1,1,2,2,3,3,4,4,5,5-undecafluorooctane, 1,1,1,2,2,3,3,4,4,5,5,6,6,7,7-pentadecafluorotridecane, 1-perfluorooctyloctane, 1,1,1,2,2,3,3,4,4,5,5,6,6-tridecafluorodecane, 1,1,1,2,2,3,3,4,4,13,13,14,14,15,15,16,16,16-octadecafluorohexadecane, 1,1,1,2,2,3,3,4,4,5,5,6,6,7,7,8,8-heptadecafluoropentadecane, 1,1,1,2,2,3,3,4,4,5,5,6,6,7,7,8,8,9,9,10,10,11,11,12,12-pentacosafluorohentriacontane, acetamidomethanol, 1,1,1,2,2,3,3,4,4,5,5,6,6,7,7,8,8-heptadecafluorotetradecane, 1-(henicosafluorodecyl)undecane, 1,1,1,2,2,3,3,4,4,5,5,6,6,7,7,8,8-heptadecafluorotridecane, 1,1,1,2,2,3,3,4,4,5,5,6,6,7,7,8,8,9,9,10,10,11,11,12,12-pentacosafluorooctacosane, ethylenecyanohydrin, 1,1,1,2,2,3,3,4,4,5,5,6,6,7,7,8,8,9,9,10,10-henicosafluoroicosane, 1,1,1,2,2,3,3,4,4,5,5,6,6-tridecafluorooctane, perfluorooctylbromide, 1,1,1,2,2,3,3,4,4,5,5,6,6,7,7,8,8,9,9,10,10-henicosafluorooctadecane, 1,1,1,2,2,3,3,4,4,5,5,6,6,7,7,8,8-heptadecafluorododecane, 1,1,1,2,2,3,3,4,4,5,5,6,6,7,7,8,8,9,9,10,10,11,11,12,12-pentacosafluorotetracosane, pentacosafluorotricosane, 1-(perfluorohexyl)propane, 1,1,1,2,2,3,3,4,4,5,5,6,6,7,7,8,8,9,9,10,10-henicosafluoropentadecane, 1,1,1,2,2,3,3,4,4,5,5,6,6,7,7,8,8-heptadecafluorodecane, 1,1,1,2,2,3,3,4,4,5,5,6,6,7,7,8,8,9,9,10,10-henicosafluorotetradecane, 1,1,1,2,2,3,3,4,4,5,5,6,6,7,7,8,8,9,9,10,10,11,11,12,12-pentacosafluorooctadecane, 1,1,1,2,2,3,3,4,4,5,5,6,6,7,7,8,8,9,9,10,10-henicosafluorotridecane, 1,1,1,2,2,3,3,4,4,5,5,6,6,7,7,8,8,9,9,10,10-henicosafluorododecane, glycol, tetratriacontafluorotetracosane, glycerol, 1,1,1,2,2,3,3,4,4,5,5,6,6,7,7,8,8,9,9,10,10,11,11,12,12-pentacosafluorotetradecane, methylhydroperoxide, H₂O, 4-hydroxymethyl-1,3-dioxolan-2-one, hydroxyacetonitrile, perfluorooctane, perfluorodecalin, methanesulfonicacid, formamide, 4-methylphenol, PBr₃, H₂O₂, BBr₃, methylbutane, 2,2-dimethylbutane, 2,2,3-trimethyl-butane, 2,2-dimethylpentane, 2,3-dimethylbutane, pentane, 2,4-dimethylpentane, 2-methylpentane, 2-methylhexane, 3-methylpentane, 3-methylhexane, 3-ethylpentane, 3-ethyl-3-methylpentane, 2-methylheptane, 3,3-diethylpentane, 3-methylheptane, 2-methyloctane, 4-methylheptane, 3-ethylheptane, 2-methyl-nonane, 3-methyloctane, 4-methyloctane, 2-methyldecane, 3-methyl-nonane, 1,1,3,3-tetramethylcyclopentane, 1,3-butanediol, 4-methyldecane, 5-methyl-nonane, 4-methyl-nonane, 1,1-dimethylcyclopentane, 1,1,2-trimethylcyclopentane, 1,1,2-trimethylcyclohexane, n-decane, n-nonane, isobutylcyclopentane, isopropylcyclopentane, tert-butylcyclohexane, pentadecane, tridecane, 1,1,3-trimethylcyclopentane, 1,1,3-trimethylcyclohexane, nonadecane, heptadecane, octadecane, ethylcyclopentane, dodecane, n-undecane, propylcyclopentane, isobutylcyclohexane, isopropylcyclohexane, 1-ethyl-1-methylcyclopentane, 2,3,3-trimethyl-1-butene, 2,4,4-trimethyl-1-pentene, n-hexylcyclopentane, pentylcyclopentane, 1-isopropyl-4-methylcyclohexane, trimethylaluminum, n-tetradecylcyclopentane, tridecylcyclopentane, tetradecane, n-dodecylcyclopentane, n-decylcyclopentane, n-propylcyclohexane, ethylcyclohexane, 3,3-dimethyl-1-butene, n-hexadecane, pentene, 2,3,3-trimethyl-1-pentene, decylcyclohexane, pentylcyclohexane, 3,4-dicyclohexyl-2,5-dimethylhexane, 2-methyl-1-butene, 3,3-dimethyl-1-pentene, 4,4-dimethyl-1-pentene, butylcyclohexane, methylcyclopentane, methylcyclohexane, decahydronaphthalene, hexahydroindene, bicyclohexyl, cyclohexane, 2,4,4-trimethyl-2-pentene, 2,3-dimethyl-1-pentene, tricyclohexylmethane, butylcyclopentane, 1-hexene, 2,3-dimethyl-1-butene, methylformamide, 3,3-dimethyl-1-butyne, triethyleneglycol, 3-methoxy-1,2-propanediol, 2,4-dimethyl-1-pentene, 1-heptene, 3-methyl-1-pentene, 3-ethyl-2-methyl-1-pentene, 1,4-di-tert-butylbenzene, diethyleneglycol, octane, methoxyaceticacid, 1-nonene, 4-methyl-1-hexene, 1,1,3,4,4,6-hexamethyltetralin, 1,3,5-triisopropylbenzene, 1,2,2,6,6-pentamethylpiperidine, 1,4-diethylbenzene, 2-methyl-2-butene, isoprene, 4-methyl-1-pentene, 1-tetradecene, 3-ethyl-1-pentene, 2-methyl-3-heptene, 1-decene, 2,3,4-trimethyl-2-pentene, 1-nonadecene, butylbenzene, 2-ethoxy-2-methyl-propane, 1,2-diethylbenzene, ethanedial, cyclohexylbenzene, galaxolide, 1,2,3,4-tetramethylbenzene, 1,2,3,5-tetramethylbenzene, pentaethylbenzene, 1,3,5-trimethylbenzene, isobutanol, peroxyaceticacid, 1,6-hexanediol, 2,6-di-tert-butyl-4-methylpyridine, 2,2′-(methylimino)bis-ethanol, 1,3-bis(1-methylethyl)-benzene, 2,7-diisopropylnaphthalene, 1,2,3,5-tetraethylbenzene, tetrahydro-2,2,5,5-tetramethylfuran, 1,5-diisopropylnaphthalene, 2,3-butanediol, 1,2-diisopropylbenzene, 1-(2-pentyl)-4-(3-pentyl)benzene, tetradecylbenzene, pentadecylbenzene, ethylbenzene, 3,6-bis-(methoxymethyl)-durene, 5-hydroxy-6-methoxy-8-[(4-amino-1-methylbutyl)amino]quinoline trihydrobromide, 1-(1,1-dimethylethyl)-3-methylbenzene, tridecylbenzene, 1-(methoxymethyl)-4-(2,4,4-trimethylpentan-2-yl)benzene, dodecylbenzene, 1,2,4-trimethylbenzene, 1-cyclohexyl-2-isopropylbenzene, 4,6,6-Trimethylbicyclo(3,1,1)hept-3-ene, 2,6-di-tert-butylpyridine, 1,1′-oxybis-octane, 1,5-hexanediol, 1-(1,1-dimethylethyl)-3,5-dimethylbenzene, menthylisovalerate, 1-methyl-7-(1-methylpropyl)naphthalene, tert-butanol, beta-himachalen, dimethoxymethane, 1,2,3,4,5,6,7,8-octahydroanthracene, tert-butylbenzene, 1,2-hexanediol, cyclohexene, 6,10,14-trimethylpentadecan-2-one, delta-cadinene, isopropylbenzene, undecylbenzene, 2,5-hexanediol, dipropyleneglycol, 1,6-dimethyl-4-isopropyltetralin, di-n-propylether, diisopropylether, cuparene, 1,4-bis(1-methylethyl)-benzene, 2,4-dimethyl-3-thiapentane, 1-methoxy-4-(2,4,4-trimethylpentan-2-yl)benzene, 1-ethoxybutane, decylbenzene, or cycloheptane, but is not limited thereto.

Yet another exemplary embodiment of the present specification provides recycled polyvinyl chloride obtained by filtering the above-described composition.

In another exemplary embodiment of the present specification, for polyvinyl chloride extracted by the good solvent from waste including polyvinyl chloride in the waste polyvinyl chloride recycled composition, most of polyvinyl chloride may be precipitated by the non-solvent and settled in a solid state. Recycled polyvinyl chloride may be obtained by filtering the composition including polyvinyl chloride in the precipitated state.

In still another exemplary embodiment of the present specification, recycled polyvinyl chloride includes all the states in which the composition has been filtered and in which excess solvents have been removed by drying the composition after filtration.

In an exemplary embodiment of the present specification, the recycled polyvinyl chloride includes an additive derived from waste. In this case, the additive derived from waste is not particularly limited as long as the additive is added for preparing the waste, and examples thereof include a plasticizer, and the like. Since an additive such as a plasticizer is already included when products are produced using the recycled polyvinyl chloride, there is an advantage in that the additive such as a plasticizer is not added or the addition amount can be reduced.

In an exemplary embodiment of the present specification, the waste is not particularly limited as long as the waste includes PVC, and may be, for example, a building material (a pipe, a window frame, and the like), an electric wire coating, synthetic leather, a rubber tub, a hose, a flooring material, a packing material, a medical sap bag, a toy, or the like, which include PVC.

In an exemplary embodiment of the present specification, the recycled polyvinyl chloride is PVC extracted by the solvent extraction, and low-molecular weight PVCs are filtered out during solvent extraction, resulting in a higher average molecular weight than PVC prepared by polymerization or PVC in waste. Furthermore, as the average molecular weight of PVC is changed, dynamic physical properties affected by the average molecular weight such as the glass transition temperature (Tg) and melting temperature (Tm) thereof are changed, so dynamic physical properties such as transition temperature (Tg) and melting temperature (Tm) are also increased as the average molecular weight of PVC is increased.

### EXAMPLES

Hereinafter, the present specification will be described in more detail through Examples. However, the following Examples are provided only for exemplifying the present specification, but are not intended to limit the present specification.

### EXAMPLES

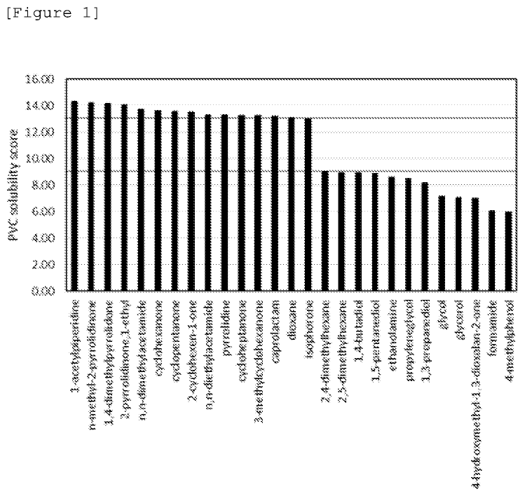

The PVC solubility score values for the solvents in the following Table 1 were derived and are shown in Table 1, and the PVC solubility score values for some of them are shown in FIG. 1. In this case, x₁ is 1, x₂ is 0.005, x₃ is 0.04, x₄ is 10, x₅ is 0.03, x₆ is 0.003, x₇ is 0.001, x₈ is 0.0001, and x₉ is 0.

The control variables x₁ to x₉ were calculated by Equation 1 in the following Document #1 (Grause et al, J. Mater. Cycles Waste Manag., 2017, 19, 612), and were adjusted so as to match the tendency of the Q (swelling ratio) values described in Table 1 of Document #1.

μ_{pvc}^{sol} and μ_{dop}^{sol} were obtained by calculating a molecular surface charge distribution using the Turbomole program based on the solvent, pvc, and dop molecular structures, and through this, the corresponding values were calculated using the COSMOtherm program, and Vₛₒₗ was also calculated through the COSMOtherm based on the structure of the solvent.

For r_{pvc} and r_{dop}, each value was calculated by substituting a Hansen solubility parameter result value derived by inputting each molecular structure into the HSPip program in SMILES form into Equation 2.

TABLE 1

| | | | | | | | PVC |
|---|---|---|---|---|---|---|---|
| | B.P._EXP | | | | | | solubility |
| Name | [ ° C.] | μ_{pvc}^{sol} | μ_{dop}^{sol} | vₛₒₗ | r_{pvc} | r_{dop} | score |
| 1,2,3-propanetrioltrinitrate | 250 | −15.48 | −10.22 | 252.12 | 5.84 | 9.86 | 14.70 |
| tetramethylenesulfoxide | 236 | −14.89 | −6.49 | 139.83 | 2.16 | 7.89 | 14.49 |
| 1-piperidinecarboxaldehyde | 222.5 | −13.76 | −6.78 | 184.54 | 0.91 | 7.26 | 14.37 |
| 1-acetylpiperidine | 226.5 | −13.82 | −6.90 | 209.97 | 1.30 | 5.92 | 14.34 |
| n-methyl-2-pyrrolidinone | 202 | −14.58 | −6.92 | 159.19 | 2.99 | 7.26 | 14.21 |
| 1,4-dimethylpyrrolidone | 215.78 | −14.20 | −6.91 | 188.00 | 2.67 | 4.15 | 14.19 |
| 1,3-dimethyl-2-imidazolidinone | 225 | −13.49 | −6.60 | 181.10 | 1.08 | 6.52 | 14.19 |
| 1,5-dimethylpyrrolidone | 217 | −14.24 | −6.93 | 189.21 | 2.85 | 3.98 | 14.18 |
| hexamethylphosphoramide | 235 | −14.04 | −7.02 | 310.81 | 2.18 | 8.18 | 14.17 |
| 1,3-dimethytetrahydropyrimidin-2(1h)-one | 246 | −13.73 | −6.82 | 201.81 | 1.85 | 5.22 | 14.15 |
| 2-pyrrolidinone,1-ethyl | 203.561 | −14.18 | −6.93 | 188.80 | 2.78 | 6.93 | 14.08 |
| 1,3-dimethylpyrrolidone | 215.78 | −13.93 | −6.90 | 190.47 | 2.85 | 3.98 | 14.02 |
| 4-acetylmorpholine | 245.5 | −12.88 | −5.98 | 188.71 | 0.81 | 6.44 | 13.89 |
| 1-[ethoxy(methyl)phosphoryl]methylpiperidine | 264.73 | −12.76 | −6.55 | 321.78 | 1.09 | 6.79 | 13.85 |
| n,n-dimethylpropanamide | 174.5 | −13.76 | −6.87 | 189.77 | 3.28 | 5.73 | 13.77 |
| n,n-dimethylacetamide | 166 | −14.44 | −6.85 | 161.05 | 4.54 | 7.75 | 13.71 |
| tributylphosphine oxide | 347.668 | −13.40 | −6.85 | 406.61 | 3.08 | 4.18 | 13.70 |
| n-n-propylpyrrolidone | 239.77 | −13.79 | −6.91 | 217.35 | 3.94 | 2.93 | 13.69 |
| n-isopropylpyrrolidone | 228.74 | −13.92 | −6.92 | 219.27 | 4.42 | 2.44 | 13.64 |
| cyclohexanone | 155.6 | −13.27 | −6.98 | 168.22 | 3.42 | 3.42 | 13.60 |
| n,n,4-trimethylbenzamide | 287.88901 | −12.06 | −6.39 | 265.30 | 0.90 | 7.17 | 13.59 |
| cyclopentanone | 131 | −13.48 | −6.92 | 144.64 | 3.69 | 5.93 | 13.54 |
| n-methyl-o-acetotoluidide | 260.61099 | −12.02 | −6.36 | 271.20 | 1.08 | 6.66 | 13.53 |
| 2-cyclohexen-1-one | 168 | −12.97 | −6.56 | 156.81 | 2.98 | 3.97 | 13.49 |
| n,n,3-trimethylbenzamide | 289.595 | −12.04 | −6.39 | 266.66 | 1.32 | 6.58 | 13.48 |
| n,n-dimethylbenzamide | 256.06 | −11.85 | −6.14 | 238.18 | 0.94 | 6.45 | 13.48 |
| thiacyclobutane | 95 | −12.58 | −7.22 | 122.90 | 2.56 | 7.73 | 13.46 |
| n,n,2-trimethylbenzamide | 286.109 | −11.85 | −6.31 | 269.53 | 1.08 | 6.66 | 13.45 |
| 1,3-dioxane | 106.1 | −13.03 | −6.56 | 133.39 | 3.32 | 4.46 | 13.41 |
| tetramethylurea | 175 | −14.20 | −7.03 | 209.56 | 5.40 | 7.99 | 13.41 |
| 2-azacyclooctanone | 287.62399 | −11.68 | −5.86 | 198.74 | 0.88 | 6.28 | 13.39 |
| 4-methyl-1-propyl-2-pyrrolidinone | 252.33 | −13.58 | −6.91 | 245.17 | 4.86 | 2.34 | 13.37 |
| 5-methyl-1-propyl-2-pyrrolidinone | 252.33 | −13.61 | −6.93 | 245.07 | 5.09 | 2.33 | 13.33 |
| n,n-diethylacetamide | 185.49899 | −13.70 | −6.87 | 219.37 | 4.87 | 6.17 | 13.32 |
| 1,3-propanedithiol | 169 | −12.43 | −6.78 | 160.81 | 2.89 | 5.12 | 13.30 |
| 4,5-dihydro-2-methylthiazole | 144.4 | −12.35 | −6.53 | 147.17 | 2.61 | 5.28 | 13.30 |
| pyrrolidine | 86.5 | −12.79 | −6.97 | 136.86 | 3.59 | 5.05 | 13.30 |
| 3-methoxy-n,n-dimethylpropanamide | 202.57 | −13.19 | −6.54 | 224.23 | 3.70 | 7.59 | 13.29 |
| 4-fluoro-n,n-dimethylbenzamide | 269.44901 | −11.19 | −5.98 | 245.36 | 0.81 | 6.86 | 13.24 |
| 3-methyl-1-propyl-2-pyrrolidinone | 252.33 | −13.41 | −6.93 | 250.25 | 5.09 | 2.33 | 13.24 |
| cycloheptanone | 179 | −13.01 | −6.96 | 194.02 | 4.27 | 4.16 | 13.23 |
| 1-morpholinopentan-1-one | 298.318 | −12.26 | −6.37 | 273.58 | 2.71 | 4.70 | 13.22 |
| 3-methylcyclohexanone | 169 | −12.83 | −6.92 | 198.32 | 4.12 | 2.81 | 13.22 |
| n,n-dimethylbutanamide | 186 | −13.34 | −6.88 | 218.13 | 4.70 | 5.62 | 13.22 |
| caprolactam | 272.48199 | −11.71 | −5.68 | 173.05 | 1.36 | 7.11 | 13.22 |
| tetrahydrothiophene | 121 | −12.40 | −7.19 | 143.71 | 3.45 | 4.95 | 13.21 |
| 2-methoxy-n,n-dimethylbenzamide | 316.89801 | −11.67 | −6.00 | 278.53 | 1.39 | 8.15 | 13.21 |
| dimethylformamide | 153 | −14.38 | −6.65 | 135.29 | 5.98 | 10.71 | 13.21 |
| 1,2-ethanedithiol | 146 | −12.51 | −6.58 | 132.23 | 3.13 | 6.18 | 13.20 |
| 1,4,7,10-tetraoxacyclododecane | 249.65 | −12.22 | −6.34 | 249.33 | 2.63 | 5.75 | 13.20 |
| ethyl1,3-dimethyl-2-oxopyrrolidine-3-carboxylate | 278.973 | −12.47 | −6.31 | 283.32 | 3.15 | 4.41 | 13.19 |
| 1-(4-methylpiperidin-1-yl)butan-1-one | 274.34 | −12.60 | −6.85 | 297.88 | 3.88 | 2.98 | 13.17 |
| 2-methylcyclohexanone | 163.69099 | −12.71 | −6.96 | 198.02 | 4.18 | 2.68 | 13.17 |
| 1-(2,6-dimethylmorpholin-4-yl)butan-1-one | 296.639 | −12.30 | −6.54 | 303.34 | 3.16 | 3.88 | 13.17 |
| n,n-diethylformamide | 176 | −13.63 | −6.76 | 193.27 | 5.13 | 7.53 | 13.16 |
| 4-(propan-2-yl)phenyldimethylcarbamate | 280.294 | −11.18 | −6.51 | 336.77 | 1.51 | 5.55 | 13.15 |
| n,n-dimethyl-3-phenylpropanamide | 294.737 | −11.63 | −6.27 | 292.94 | 2.01 | 5.10 | 13.15 |
| Metolachlor | 406.819 | −10.96 | −6.45 | 445.58 | 1.18 | 5.78 | 13.15 |
| dichloroacetylchloride | 108 | −11.24 | −7.97 | 162.72 | 2.58 | 4.25 | 13.14 |
| n,n-diethyl-3-(2-methyl-1,3-dioxolan-2-yl)propanamide | 306.953 | −12.21 | −6.47 | 345.40 | 3.11 | 4.34 | 13.12 |
| n-acetylcaprolactam | 247 | −12.25 | −6.45 | 234.43 | 3.09 | 5.19 | 13.12 |
| d-camphor | 204 | −12.05 | −6.85 | 265.91 | 3.14 | 4.59 | 13.11 |
| n-methylformanilide | 243 | −10.88 | −5.72 | 209.11 | 0.75 | 6.85 | 13.11 |
| ethyl-3-acetyl(ethyl)amino-propanoate | 281.834 | −12.40 | −6.28 | 304.23 | 3.30 | 4.77 | 13.11 |
| (2,5-dimethoxy-phenyl)acetaldehyde | 289.51 | −10.65 | −5.90 | 270.46 | 0.67 | 6.20 | 13.10 |
| 4-methylcyclohexanone | 171 | −12.78 | −6.91 | 198.06 | 4.53 | 2.62 | 13.10 |
| 2,6-dimethyl-2,5-cyclohexadiene-1,4-dione | 200.892 | −11.57 | −6.18 | 205.57 | 1.88 | 7.20 | 13.09 |
| dioxane | 101.2 | −12.95 | −6.49 | 133.17 | 4.32 | 4.81 | 13.09 |
| 3-oxiranyl-7-oxabicyclo[4,1,0]heptane | 227 | −12.32 | −6.45 | 208.28 | 3.42 | 4.22 | 13.09 |
| 1-butyl-4-methyl-2-pyrrolidinone | 269.25 | −13.28 | −6.89 | 271.78 | 5.46 | 2.31 | 13.08 |
| nicotine | 247 | −11.46 | −6.73 | 263.46 | 2.28 | 5.16 | 13.08 |
| 2-methylcyclopentanone | 139 | −12.89 | −6.94 | 174.82 | 4.87 | 2.01 | 13.08 |
| dimethylveronal | 275.816 | −11.63 | −6.39 | 321.70 | 2.39 | 4.63 | 13.08 |
| diethyl(trichloromethyl)phosphonate | 259.961 | −11.53 | −6.45 | 318.63 | 2.28 | 4.74 | 13.07 |
| pulegone | 224 | −12.02 | −6.88 | 269.38 | 3.35 | 3.55 | 13.07 |
| 1-heptanoyl-4-methylpiperazine | 324.55 | −12.06 | −6.70 | 365.51 | 3.29 | 3.65 | 13.07 |
| n,n-diethyl-4-oxopentanamide | 275.794 | −12.51 | −6.38 | 296.57 | 3.62 | 5.02 | 13.07 |
| n-ethyl-n-phenylacetamide | 245.364 | −11.78 | −6.35 | 269.07 | 2.58 | 4.72 | 13.07 |
| dimethyldisulfide | 109.8 | −11.91 | −6.86 | 147.43 | 3.14 | 4.02 | 13.07 |
| 4,6-dimethylpyrimidine | 154 | −11.92 | −6.44 | 173.72 | 2.91 | 4.06 | 13.07 |
| 1,4-oxathiane | 147 | −12.29 | −6.40 | 145.21 | 3.16 | 6.66 | 13.07 |
| n-sec-butylpyrrolidone | 246.85 | −13.23 | −6.88 | 247.86 | 5.44 | 2.10 | 13.07 |
| 1-(4-methylpiperidin-1-yl)pentan-1-one | 291.23 | −12.50 | −6.84 | 325.22 | 4.15 | 2.72 | 13.07 |
| 1-butyl-5-methyl-2-pyrrolidinone | 269.25 | −13.32 | −6.92 | 274.27 | 5.61 | 2.51 | 13.06 |
| 3-methyl-2-oxazolidone | 266.2 | −12.65 | −5.52 | 141.34 | 2.98 | 8.81 | 13.05 |
| n,n-dimethyl-4-(propan-2-yl)benzamide | 304.765 | −11.90 | −6.54 | 322.17 | 2.97 | 4.40 | 13.05 |
| 2,5-dimethylpyrazine | 155 | −11.79 | −6.51 | 176.69 | 2.91 | 4.06 | 13.03 |
| mesitenelactone | 244.99899 | −12.03 | −6.02 | 185.44 | 2.87 | 4.94 | 13.03 |
| propyl4-(dimethylamino)-4-oxobutanoate | 279.263 | −12.16 | −6.33 | 307.47 | 3.23 | 5.55 | 13.02 |
| diethyldimethylamidophosphate | 232.05 | −12.59 | −6.72 | 291.62 | 4.13 | 5.46 | 13.02 |
| isophorone | 215.32 | −12.71 | −6.74 | 243.63 | 4.66 | 2.29 | 13.02 |
| n,n-dimethyl4-methoxybenzamide | 315.41599 | −11.55 | −5.99 | 273.08 | 1.93 | 8.71 | 13.00 |
| 2,4-dimethylhexane | 109.5 | −7.91 | −6.06 | 276.28 | 14.64 | 8.46 | 8.99 |
| 3-ethyl-2,4-dimethylpentane | 136.7 | −7.91 | −6.06 | 302.65 | 14.64 | 8.46 | 8.98 |
| 2,7-dimethyloctane | 159.9 | −7.77 | −5.97 | 330.60 | 14.43 | 8.30 | 8.98 |
| 2,6-dimethyl-heptane | 135.2 | −7.82 | −6.00 | 303.63 | 14.53 | 8.38 | 8.98 |
| 2-methyl-3-ethylpentane | 115.6 | −7.87 | −6.05 | 275.31 | 14.64 | 8.46 | 8.98 |
| 3,3-dimethylhexane | 111.9 | −7.94 | −6.08 | 275.51 | 14.74 | 8.55 | 8.97 |
| 2,3,5-trimethylhexane | 131.4 | −7.87 | −6.04 | 307.28 | 14.64 | 8.46 | 8.97 |
| 2,3,4-trimethylpentane | 113.5 | −7.92 | −6.07 | 275.61 | 14.74 | 8.55 | 8.97 |
| 2,2-dimethylheptane | 132.7 | −7.86 | −6.01 | 303.48 | 14.64 | 8.46 | 8.97 |
| 3,3-dimethylpentane | 86 | −8.04 | −6.14 | 246.19 | 14.96 | 8.73 | 8.96 |
| 1,1,1,2,2,3,3,4,4-nonafluorotetracosane | 363.41 | −7.36 | −5.80 | 799.59 | 14.00 | 7.84 | 8.96 |
| 2,5-dimethylhexane | 109.1 | −7.88 | −6.04 | 276.33 | 14.74 | 8.55 | 8.95 |
| 1,4-butadiol | 235 | −8.65 | −4.46 | 153.57 | 13.91 | 18.24 | 8.95 |
| 2,2-dimethylhexane | 106.8 | −7.94 | −6.07 | 275.52 | 14.85 | 8.64 | 8.95 |
| 2,3,3-trimethylpentane | 114.8 | −7.97 | −6.10 | 273.02 | 14.96 | 8.73 | 8.94 |
| Formic acid | 101 | −5.25 | −3.11 | 66.98 | 10.35 | 11.99 | 8.94 |
| 2,2,6-trimethylheptane | 148.9 | −7.83 | −6.00 | 332.54 | 14.74 | 8.55 | 8.94 |
| 2,2,4-trimethylhexane | 126.5 | −7.88 | −6.03 | 304.79 | 14.85 | 8.64 | 8.93 |
| 2,2,3-trimethylpentane | 110 | −8.02 | −6.12 | 273.29 | 15.08 | 8.83 | 8.93 |
| 2,3,3,4-tetramethylpentane | 141.5 | −7.93 | −6.09 | 299.62 | 14.96 | 8.73 | 8.93 |
| 2,4,4-trimethylhexane | 130.7 | −7.86 | −6.03 | 304.11 | 14.85 | 8.64 | 8.92 |
| 1-hydroxybenzotriazole | 284.99 | −6.17 | −4.68 | 161.29 | 11.32 | 17.75 | 8.92 |
| 3-ethyl-2,2-dimethyl-pentane | 133.8 | −7.90 | −6.06 | 303.56 | 14.96 | 8.73 | 8.92 |
| 2,2,4,6,6-pentamethylheptane | 177.8 | −7.84 | −6.00 | 393.97 | 14.85 | 8.64 | 8.92 |
| 2,2,5-trimethylhexane | 124 | −7.91 | −6.04 | 302.22 | 14.96 | 8.73 | 8.92 |
| 2-nitroethanol | 194 | −6.70 | −2.79 | 120.93 | 11.21 | 16.40 | 8.91 |
| 2,2,3,4-tetramethylpentane | 133 | −7.96 | −6.08 | 304.45 | 15.08 | 8.83 | 8.91 |
| octamethyltrisiloxane | 153 | −9.20 | −6.45 | 474.67 | 17.05 | 10.68 | 8.91 |
| 2,2,3,3-tetramethylhexane | 160.3 | −7.88 | −6.04 | 326.93 | 15.08 | 8.83 | 8.89 |
| 1,5-pentanediol | 239 | −7.30 | −4.16 | 179.17 | 12.59 | 16.84 | 8.88 |
| 1,1,1,2,2,3,3-heptafluoroundecane | 135.66 | −7.11 | −5.57 | 412.25 | 14.03 | 7.79 | 8.87 |
| 2,2,5,5-tetramethylhexane | 137.4 | −7.90 | −6.03 | 332.85 | 15.19 | 8.93 | 8.87 |
| 2,2,3,3-tetramethylpentane | 140.2 | −7.96 | −6.09 | 299.48 | 15.31 | 9.04 | 8.87 |
| 2,2,4-trimethylpentane | 99.2 | −7.98 | −6.09 | 278.43 | 15.43 | 9.14 | 8.85 |
| 2,2,4,4-tetramethylpentane | 122.2 | −7.98 | −6.08 | 306.03 | 15.43 | 9.14 | 8.85 |
| Dodecamethylcyclohexasiloxane | 245 | −9.25 | −6.48 | 779.68 | 17.48 | 11.35 | 8.84 |
| acetaldehydeammonia | 110 | −7.02 | −3.28 | 105.10 | 12.12 | 16.58 | 8.84 |
| 1,1,1,2,2,3,3,4,4-nonafluorohexadecane | 251.98 | −7.00 | −5.56 | 572.46 | 14.17 | 7.94 | 8.81 |
| dodecamethylpentasiloxane | 232 | −8.99 | −6.39 | 740.24 | 17.24 | 11.01 | 8.80 |
| 1,1,1,2,2,3,3-heptafluorononane | 88.56 | −6.95 | −5.44 | 357.26 | 14.11 | 7.89 | 8.80 |
| 1,3-dioxolan-2-one | 248 | −7.46 | −1.70 | 105.64 | 12.36 | 15.10 | 8.77 |
| decamethyltetrasiloxane | 194 | −9.01 | −6.38 | 614.16 | 17.57 | 11.26 | 8.75 |
| 1-(perfluorohexyl)docosane | 411.11 | −6.96 | −5.59 | 942.99 | 14.65 | 8.43 | 8.70 |
| hexamethyldisilane | 113.5 | −8.14 | −6.17 | 341.87 | 16.43 | 10.28 | 8.70 |
| 1,1,1,2,2,3,3,4,4,5,5-undecafluorononadecane | 300.9 | −6.80 | −5.46 | 674.29 | 14.48 | 8.25 | 8.69 |
| tetradecamethylhexasiloxane | 245.5 | −8.87 | −6.36 | 867.43 | 17.67 | 11.43 | 8.68 |
| 1,1,1,2,2,3,3,4,4,5,5,6,6-tridecafluorohexacosane | 387.9 | −6.89 | −5.54 | 885.97 | 14.70 | 8.46 | 8.67 |
| 1,1,1,2,2,3,3,4,4-nonafluoroundecane | 153.72 | −6.64 | −5.29 | 432.03 | 14.36 | 8.10 | 8.66 |
| 1,1,1,2,2,3,3,4,4-nonafluorododecane | 175.18 | −6.70 | −5.33 | 458.86 | 14.45 | 8.21 | 8.66 |
| hexadecamethylheptasiloxane | 270 | −9.06 | −6.40 | 999.44 | 18.10 | 11.85 | 8.66 |
| 1,1,1,2,2,3,3,4,4,5,5,6,6-tridecafluorotetracosane | 364.69 | −6.88 | −5.54 | 834.10 | 14.84 | 8.59 | 8.64 |
| propynol | 113.6 | −5.92 | −3.76 | 104.34 | 12.30 | 15.82 | 8.63 |
| 1,1,1,2,2,3,3,4,4-nonafluorodecane | 131.36 | −6.51 | −5.19 | 403.39 | 14.41 | 8.19 | 8.62 |
| ethanolamine | 171 | −8.38 | −3.76 | 104.16 | 14.90 | 19.83 | 8.60 |
| 1,1,1,2,2,3,3,4,4,5,5,6,6-tridecafluorodocosane | 341.38 | −6.80 | −5.48 | 775.72 | 14.95 | 8.69 | 8.60 |
| 2-hydroxypropanenitrile | 183 | −5.75 | −2.82 | 118.71 | 11.85 | 16.90 | 8.59 |
| 1,1,1,2,2,3,3,4,4,5,5,6,6-tridecafluoroicosane | 315.84 | −6.68 | −5.38 | 719.48 | 14.91 | 8.64 | 8.57 |
| 1,1,1,2,2,3,3,4,4-nonafluorononane | 108.09 | −6.29 | −5.04 | 375.91 | 14.62 | 8.40 | 8.51 |
| propyleneglycol | 187.6 | −7.19 | −3.91 | 127.81 | 14.14 | 18.52 | 8.50 |
| 1,1,1,2,2,3,3,4,4,5,5,6,6-tridecafluorooctadecane | 286.68 | −6.44 | −5.24 | 662.07 | 15.08 | 8.79 | 8.47 |
| cis-2-butene-1,4-diol | 235 | −7.22 | −3.69 | 142.23 | 14.29 | 19.31 | 8.45 |
| 1,1,1,2,2,3,3,4,4,5-decafluorononane | 106.29 | −6.32 | −5.10 | 381.33 | 15.02 | 8.79 | 8.44 |
| 1,1,1,2,2,3,3,4,4,5,5,6,6,7,7,8,8- | 412.39 | −6.65 | −5.42 | 977.43 | 15.57 | 9.29 | 8.43 |
| heptadecafluorooctacosane | | | | | | | |
| 1,1,1,2,2,3,3,4,4,5,5,6,6-tridecafluorohexadecane | 253.9 | −6.25 | −5.11 | 608.17 | 15.06 | 8.80 | 8.42 |
| 1,1,1,2,2,3,3,4,4,5,5,6,6,7,7,8,8- | 389.18 | −6.52 | −5.33 | 927.07 | 15.60 | 9.32 | 8.39 |
| heptadecafluorohexacosane | | | | | | | |
| 1,1,1,2,2,3,3,4,4,5,5,6,6,7,7,8,8- | 377.58 | −6.45 | −5.29 | 897.94 | 15.60 | 9.32 | 8.37 |
| heptadecafluoropentacosane | | | | | | | |
| 1,1,1,2,2,3,3,4,4,5,5,6,6-tridecafluoropentadecane | 236.16 | −6.14 | −5.03 | 578.97 | 15.19 | 8.93 | 8.36 |
| 1,1,1,2,2,3,3,4,4,5,5,6,6,7,7,8,8- | 365.98 | −6.37 | −5.23 | 870.01 | 15.70 | 9.42 | 8.33 |
| heptadecafluorotetracosane | | | | | | | |
| 1-(perfluorohexyl)octane | 320.00 | −6.03 | −4.94 | 548.39 | 15.24 | 8.96 | 8.32 |
| 1,1,1,2,2,3,3,4,4,5,5-undecafluorodecane | 132.62 | −5.77 | −4.73 | 421.41 | 14.97 | 8.72 | 8.31 |
| 1,1,1,2,2,3,3,4,4,5,5,6,6-tridecafluorotridecane | 197.96 | −5.85 | −4.82 | 522.67 | 15.25 | 8.98 | 8.27 |
| 1,1,1,2,2,3,3,4,4,5,5,6,6,7,7,8,8- | 342.69 | −6.18 | −5.10 | 815.81 | 15.86 | 9.59 | 8.24 |
| heptadecafluorodocosane | | | | | | | |
| 1h,8h-hexadecafluorooctane | 132 | −4.77 | −5.31 | 397.59 | 14.61 | 8.68 | 8.23 |
| 1,1,1,2,2,3,3,4,4,5,5,6,6,7,7,8,8,9,9,10,10- | 436.88 | −6.34 | −5.24 | 1074.76 | 16.30 | 10.03 | 8.20 |
| henicosafluorotriacontane | | | | | | | |
| Hexacosamethyldodecasiloxane | 280 | −8.89 | −6.45 | 1701.16 | 20.46 | 14.31 | 8.20 |
| 1,1,1,2,2,3,3,4,4,5,5,6,6,7,7,8,8- | 317.34 | −6.00 | −4.98 | 759.03 | 15.91 | 9.62 | 8.18 |
| heptadecafluoroicosane | | | | | | | |
| 1,1,1,2,2,3,3,4,4,5,5,6,6-tridecafluorododecane | 177.5 | −5.60 | −4.66 | 493.75 | 15.40 | 9.16 | 8.18 |
| 1,3-propanediol | 211 | −7.79 | −3.78 | 125.43 | 16.41 | 21.13 | 8.18 |
| 1,1,1,2,2,3,3,4,4,5,5,6,6-tridecafluoroundecane | 156.14 | −5.39 | −4.50 | 466.13 | 15.35 | 9.10 | 8.14 |
| 1,1,1,2,2,3,3,4,4,5,5,6,6,7,7,8,8- | 303.32 | −5.85 | −4.89 | 733.09 | 16.03 | 9.74 | 8.12 |
| heptadecafluorononadecane | | | | | | | |
| 1,1,1,2,2,3,3,4,4,5,5,6,6,7,7,8,8,9,9,10,10- | 425.28 | −6.12 | −5.10 | 1049.91 | 16.46 | 10.19 | 8.11 |
| henicosafluorononacosane | | | | | | | |
| 1,1,1,2,2,3,3,4,4,5,5,6,6,7, 7,8,8- | 288.38 | −5.74 | −4.81 | 698.51 | 15.98 | 9.72 | 8.10 |
| heptadecafluorooctadecane | | | | | | | |
| 1,1,1,2,2,3,3,4,4,5,5,6,6,7,7-pentadecafluorotetradecane | 218.56 | −5.46 | −4.60 | 568.83 | 15.63 | 9.36 | 8.10 |
| 1,1,1,2,2,3,3,4,4,5,5,6-dodecafluorononane | 107.61 | −5.56 | −4.62 | 400.07 | 15.83 | 9.57 | 8.08 |
| 1,1,1,2,2,3,3,4,4,5,5-undecafluorooctane | 85.28 | −5.23 | −4.33 | 365.12 | 15.57 | 9.33 | 8.05 |
| 1,1,1,2,2,3,3,4,4,5,5,6,6,7,7-pentadecafluorotridecane | 199.06 | −5.29 | −4.47 | 541.82 | 15.78 | 9.54 | 8.03 |
| 1-perfluorooctyloctane | 255.81 | −5.45 | −4.61 | 643.15 | 16.03 | 9.74 | 8.02 |
| 1,1,1,2,2,3,3,4,4,5,5,6,6-tridecafluorodecane | 133.88 | −4.98 | −4.22 | 439.35 | 15.53 | 9.27 | 8.00 |
| 1,1,1,2,2,3,3,4,4,13,13,14,14,15,15,16,16,16- | 264.75 | −5.36 | −4.42 | 649.67 | 16.25 | 9.91 | 7.95 |
| octadecafluorohexadecane | | | | | | | |
| 1,1,1,2,2,3,3,4,4,5,5,6,6,7,7,8,8- | 238.16 | −5.18 | −4.42 | 614.91 | 16.16 | 9.89 | 7.93 |
| heptadecafluoropentadecane | | | | | | | |
| 1,1,1,2,2,3,3,4,4,5,5,6,6,7,7,8,8,9,9,10,10,11,11,12,12- | 449.77 | −5.88 | −4.96 | 1139.05 | 17.24 | 10.99 | 7.90 |
| pentacosafluorohentriacontane | | | | | | | |
| formicin | 321.34299 | −5.41 | −1.57 | 128.87 | 14.40 | 20.32 | 7.89 |
| 1,1,1,2,2,3,3,4,4,5,5,6,6,7,7,8,8- | 219.62 | −4.98 | −4.30 | 588.60 | 16.21 | 9.95 | 7.87 |
| heptadecafluorotetradecane | | | | | | | |
| 1-(henicosafluorodecyl)undecane | 331.86 | −5.47 | −4.65 | 822.27 | 16.84 | 10.57 | 7.87 |
| 1,1,1,2,2,3,3,4,4,5,5,6,6,7,7,8,8- | 200.16 | −4.78 | −4.16 | 558.68 | 16.11 | 9.86 | 7.85 |
| heptadecafluorotridecane | | | | | | | |
| 1,1,1,2,2,3,3,4,4,5,5,6,6,7,7,8,8,9,9,10,10,11,11,12,12- | 413.44 | −5.61 | −4.79 | 1063.46 | 17.19 | 10.93 | 7.84 |
| pentacosafluorooctacosane | | | | | | | |
| ethylenecyanohydrin | 221 | −4.49 | −0.65 | 114.99 | 13.78 | 18.73 | 7.82 |
| 1,1,1,2,2,3,3,4,4,5,5,6,6,7,7,8,8,9,9,10,10- | 318.84 | −5.31 | −4.56 | 788.54 | 16.89 | 10.63 | 7.82 |
| henicosafluoroicosane | | | | | | | |
| 1,1,1,2,2,3,3,4,4,5,5,6,6-tridecafluorooctane | 86.64 | −4.41 | −3.83 | 383.72 | 15.96 | 9.73 | 7.79 |
| perfluorooctylbromide | 132 | −3.53 | −3.40 | 448.83 | 15.37 | 9.17 | 7.74 |
| 1,1,1,2,2,3,3,4,4,5,5,6,6,7,7,8,8,9,9,10,10- | 290.08 | −4.93 | −4.30 | 735.91 | 16.93 | 10.66 | 7.72 |
| henicosafluorooctadecane | | | | | | | |
| 1,1,1,2,2,3,3,4,4,5,5,6,6,7,7,8,8- | 179.81 | −4.39 | −3.87 | 530.67 | 16.35 | 10.10 | 7.71 |
| heptadecafluorododecane | | | | | | | |
| 1,1,1,2,2,3,3,4,4,5,5,6,6,7,7,8,8,9,9,10,10,11,11,12,12- | 368.54 | −5.26 | −4.55 | 935.53 | 17.52 | 11.26 | 7.69 |
| pentacosafluorotetracosane | | | | | | | |
| pentacosafluorotricosane | 356.94 | −5.05 | −4.40 | 911.46 | 17.67 | 11.43 | 7.61 |
| 1-(perfluorohexyl)propane | 158.55 | −4.14 | −3.73 | 503.16 | 16.69 | 10.43 | 7.59 |
| 1,1,1,2,2,3,3,4,4,5,5,6,6,7,7,8,8,9,9,10,10- | 240.16 | −4.24 | −3.84 | 651.02 | 17.03 | 10.79 | 7.55 |
| henicosafluoropentadecane | | | | | | | |
| 1,1,1,2,2,3,3,4,4,5,5,6,6,7,7,8,8- | 136.38 | −3.72 | −3.43 | 474.73 | 16.65 | 10.42 | 7.52 |
| heptadecafluorodecane | | | | | | | |
| 1,1,1,2,2,3,3,4,4,5,5,6,6,7,7,8,8,9,9,10,10- | 221.71 | −3.88 | −3.59 | 621.30 | 17.08 | 10.82 | 7.47 |
| henicosafluorotetradecane | | | | | | | |
| 1,1,1,2,2,3,3,4,4,5,5,6,6,7,7,8,8,9,9,10,10,11,11,12,12- | 291.77 | −4.08 | −3.77 | 774.52 | 17.97 | 11.74 | 7.34 |
| pentacosafluorooctadecane | | | | | | | |
| 1,1,1,2,2,3,3,4,4,5,5,6,6,7,7,8,8,9,9,10,10- | 202.36 | −3.54 | −3.37 | 595.23 | 17.62 | 11.37 | 7.30 |
| henicosafluorotridecane | | | | | | | |
| 1,1,1,2,2,3,3,4,4,5,5,6,6,7,7,8,8,9,9,10,10- | 182.11 | −3.17 | −3.12 | 566.17 | 17.47 | 11.23 | 7.25 |
| henicosafluorododecane | | | | | | | |
| Glycol | 198 | −5.37 | −2.18 | 97.15 | 18.66 | 23.26 | 7.14 |
| Tetratriacontafluorotetracosane | 377.23 | −3.85 | −3.57 | 1027.58 | 19.15 | 12.92 | 7.08 |
| Glycerol | 290 | −5.80 | −2.48 | 130.83 | 19.74 | 24.53 | 7.05 |
| 1,1,1,2,2,3,3,4,4,5,5,6,6,7,7,8,8,9,9,10,10,11,11,12,12- | 223.8 | −2.79 | −2.90 | 659.52 | 18.22 | 12.01 | 7.05 |
| pentacosafluorotetradecane | | | | | | | |
| methylhydroperoxide | 86 | −8.05 | −4.65 | 82.40 | 24.09 | 28.25 | 7.05 |
| H₂O | 100 | 8.37 | 6.36 | 33.29 | 11.87 | 17.36 | 7.00 |
| 4-hydroxymethyl-1,3-dioxolan-2-one | 353.9 | −4.99 | −0.83 | 135.43 | 18.61 | 23.53 | 6.99 |
| hydroxyacetonitrile | 183 | −0.48 | 0.96 | 89.11 | 15.99 | 21.45 | 6.81 |
| perfluorooctane | 105.9 | −0.12 | −1.24 | 434.02 | 17.46 | 11.17 | 6.79 |
| cis-perfluorodecalin | 142 | −0.10 | −1.20 | 428.79 | 17.57 | 11.33 | 6.76 |
| methanesulfonicacid | 167 | −3.79 | −1.80 | 104.11 | 19.50 | 25.18 | 6.70 |
| formamide | 210.5 | −0.81 | 1.99 | 67.18 | 20.22 | 24.96 | 6.05 |
| 4-methylphenol | 202 | −8.98 | −7.80 | 173.65 | 39.40 | 34.07 | 5.97 |
| PBr₃ | 173.2 | −7.20 | −4.03 | 160.17 | 39.40 | 34.07 | 5.10 |
| H₂O₂ | 152 | −4.73 | −3.96 | 45.07 | 35.67 | 40.00 | 4.88 |
| BBr₃ | 90 | −6.16 | −3.71 | 153.22 | 39.40 | 34.07 | 4.85 |

Among the solvents in Table 1, the PVC solubility score values of Equation 1 and the solvent classifications of Document #1 are compared in the following Table 2 for the solvents tested for dissolution of PVC in Document #1.

TABLE 2

| | | Literature | Experimental |
|---|---|---|---|
| | PVC solubility | value | value |
| Solvent | score | (δ_{ADBP}) | (Q) |
| cyclohexanone | 13.5955 | — | Diss |
| cyclopentanone | 13.5371 | — | Diss |
| n,n- | 13.7104 | 2.006 | Diss |
| dimethylacetamide | | | |
| Tetrahydrofuran | 13.4063 | 2.11 | Diss |
| (THF) | | | |
| pyridine | 12.9354 | 1.347 | 15.04 |
| 3-pentanone | 12.5136 | — | 14.14 |
| 2-pentanone | 12.6458 | — | 12.69 |
| dimethylformamide | 13.2076 | 1.388 | 12.19 |
| butanone | 12.8923 | — | 11.53 |
| CH₂Cl₂ | 13.0545 | 5.573 | 9.47 |
| 4-methyl-2- | 11.9326 | — | 7.06 |
| pentanone | | | |
| nitrobenzene | 11.7561 | 3.64 | 6.55 |
| dioxane | 12.0292 | 3.423 | 3.88 |
| 1,1,2,2- | 12.4097 | — | 3.29 |
| tetrachloroethane | | | |
| propanone | 12.9660 | 2.086 | 2.79 |
| ethylacetate | 12.3571 | 2.778 | 2.38 |
| CHCl₃ | 12.1334 | 5.589 | 2.27 |
| dimethylsulfoxide | 12.5817 | 2.757 | 1.89 |
| toluene | 10.8072 | — | 1.21 |
| cyclohexanol | 10.4645 | — | 1.1 |
| benzene | 10.5984 | 6.533 | 1.07 |
| cyclohexane | 9.4648 | — | 1.04 |
| 1-pentanol | 10.2100 | — | 1.02 |
| H₂O | 6.9982 | 4.902 | 1 |
| methanol | 8.2364 | 3.153 | 1 |
| 1-butanol | 9.8848 | 4.254 | 1 |
| propanol | 9.6356 | 4.546 | 0.98 |
| ethanol | 9.2140 | 3.45 | 0.92 |
| hexane | 9.0600 | 10.066 | 0.77 |
| diethylether | 11.3495 | — | 0.65 |

In Table 2, the literature value (δ_{ADBP}) is the δ_{ADBP} value of Equation 4 of #1, and the experimental value (Q) is the swelling ratio value shown in Table 1 of #1. The larger the experimental value (Q), the better the solvent dissolves PVC in the experiment, and Diss completely dissolves PVC. δ_{ADBP} is used as a discriminating parameter in the corresponding literature, and when the value is less than 2.2, the solvent is determined to be a good solvent, but the trend of the corresponding value does not match the trend of the experimental value.

In Table 2, pyridine determined to be a good solvent with a δ_{ADBP} value of 1.347, which is 2.2 or less, has a value lower than that of THF with a δ_{ADBP} value of 2.11, and thus may be predicted to have a high solubility. However, THF with a δ_{ADBP} value of 2.11 in the experimental value (Q) was completely dissolved in diss, and pyridine with a δ_{ADBP} value of 1.347 showed 15.04, so it could be confirmed that pyridine is less soluble than THF.

In contrast, the higher the PVC solubility score value according to Equation 1 of the present specification, the solvent is determined as a solvent having the higher the solubility for PVC, and as an example of the reference, when the PVC solubility score value is 13 or more, the solvent may be determined as a good solvent, and when the PVC solubility score value is 9 or less, the solvent may be determined as a non-solvent.

In Table 2, the PVC solubility score value of THF according to Equation 1 is 13.4063, showing a higher value than pyridine with a PVC solubility score value of 12.9354, and when a solvent is determined by an example reference of a good solvent, THF is a good solvent, but pyridine is predicted to be a solvent with a low dissolution efficiency rather than a good solvent, confirming that the reliability of Equation 1 of the present specification is high because the value is also consistent with the experimental value.

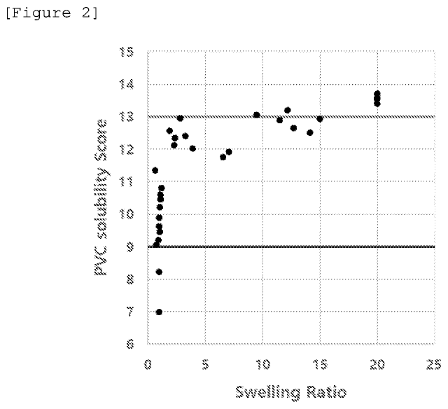

FIG. 2 is a graph showing the relationship between the PVC solubility score value calculated by Equation 1 according to the present specification and the PVC swelling ratio.

Here, the PVC swelling ratio is the value calculated by Equation 1 in #1 through experiments, and the corresponding value was extracted from Table 1 in Document #1.

### EXPERIMENTAL EXAMPLES

A solution was prepared by adding a PVC waste sample to the good solvent shown in the following Table 3 and stirring the resulting mixture at 120 rpm at room temperature for 3 hours. Thereafter, PVC was precipitated by dropping a non-solvent shown in the following Table 3 into the solution. In this case, the weight ratio of waste PVC:good solvent:non-solvent is 1:5:20, and the weight ratio of main solvent and auxiliary solvent in the good solvent is 60:40.

After all non-solvents were dropped, a filtered solid was obtained by filtering the reaction solution with a filter. The filtered solid was further washed with the same non-solvent as the above non-solvent and dried to obtain recycled PVC.

TABLE 3

| | Good solvent | | | | | PVC |
|---|---|---|---|---|---|---|
| | | Auxiliary | Non- | Sample | Recovery | recovered |
| | Main solvent | solvent | solvent | (g) | (g) | rate |
| Experimental | cyclohexanone | DMPA | IPA | 20 | 6.8 | 51% |
| Example 1 | | | | | | |
| Experimental | cyclohexanone | MC | IPA | 20 | 2.3 | 14% |
| Example 2 | | | | | | |
| Experimental | DMPA | MC | IPA | 20 | 0.9 | 7% |
| Example 3 | | | | | | |

★ DMPA: N,n-dimethylpropanamide, IPA: 2-propanol, MC: dichloromethane

PVC solubility score values for the solvents used in Table 3 are shown in the following Table 4.

Referring to Table 3 with reference to the PVC solubility score values in Table 4, in the case of dichloromethane (MC), the PVC solubility score is lowest among the good solvents used, that is, meaning that the solubility of PVC is lowest among the good solvents in the experimental examples. Therefore, it can be confirmed that Experimental Examples 2 and 3, in which dichloromethane (MC) is used as an auxiliary solvent, have recovery rates lower than that of Experimental Example 1 in which the main solvent is cyclohexanone and the auxiliary solvent is N,n-dimethylpropanamide (DMPA).

TABLE 4

| | PVC solubility score | |
|---|---|---|
| | value | Classification |
| cyclohexanone | 13.60 | Good solvent |
| DMPA | 13.77 | Good solvent |
| MC | 13.14 | Good solvent |
| IPA | 8.95 | Non-solvent |

1. A method for screening a solvent for extracting polyvinyl chloride, comprising:
screening a solvent for extracting a polyvinyl chloride from a waste including the polyvinyl chloride using the following Equation 1:
$$PVC solubility score = {x}_{1} exp ( - {x}_{2} {μ}_{pvc}^{sol} - {x}_{3} {μ}_{dop}^{sol} ) + {x}_{4} exp ( - {x}_{5} {r}_{pvc} - {x}_{6} {r}_{dop} ) - {x}_{7} exp ( - {x}_{8} {V}_{sol} ) + {x}_{9} \qquad [ Equation 1 ]$$
wherein
xₖ is each a real number from −20 to 20, and k is an integer from 1 to 9,
μᵢ^{j} is a chemical potential of the solvent, and in μᵢ^{j}, i is a solute and j is the solvent,
rᵢ is a Hansen solubility parameter distance of the solvent,
Vₛₒₗ is a molecular volume of the solvent,
sol is the solvent, and
solute is either pvc or dop, pvc is the polyvinyl chloride dop is diethylhexyl phthalate.

2. The method of claim 1, further comprising:
selecting two or more solvents with a PVC solubility score value of 11 or more according to Equation 1 as a good solvent for the polyvinyl chloride; and
selecting at least one solvent with a PVC solubility score value of less than 11 according to Equation 1 as a non-solvent for the polyvinyl chloride.

3. The method of claim 2, wherein the good solvent has a boiling point of 85° C. or more.

4. The method of claim 1, wherein x₁ is 1, x₂ is 0.005, x₃ is 0.04, x₄ is 10, x₅ is 0.03, x₆ is 0.003, x₇ is 0.001, x₈ is 0.0001, and x₉ is 0.

5. A method for recycling the waste comprising the polyvinyl chloride, comprising:
screening a solvent for extracting the polyvinyl chloride using the method of claim 1; and
extracting the polyvinyl chloride from the waste comprising the polyvinyl chloride using the screened solvent.

6. The method of claim 5, wherein the extracting of the polyvinyl chloride comprises:
a dissolution process of bringing the waste comprising the polyvinyl chloride into contact with a good solvent with a first PVC solubility score value of 11 or more according to Equation 1 and obtaining a polyvinyl chloride solution by dissolving the polyvinyl chloride in the waste in the good solvent; and
a precipitation process of precipitating the polyvinyl chloride from the polyvinyl chloride solution by bringing the polyvinyl chloride solution into contact with a non-solvent with a second PVC solubility score value of less than 11 according to Equation 1.

7. The method of claim 6, further comprising a separation process of separating a solid material from the polyvinyl chloride solution obtained by the dissolution process.

8. The method of claim 6, further comprising a rinsing process of rinsing the polyvinyl chloride precipitated by the precipitation process.

9. A recycled polyvinyl chloride extracted from the waste comprising the polyvinyl chloride using the solvent screened by the method of claim 1.

10. A waste polyvinyl chloride recycled composition comprising:
A polyvinyl chloride extracted from the waste comprising the polyvinyl chloride;
two or more good solvents with a first PVC solubility score value of 11 or more according to the following Equation 1; and
at least one non-solvent with a second PVC solubility score value of less than 11 according to the following Equation 1:
$$PVC solubility score = {x}_{1} exp ( - {x}_{2} {μ}_{pvc}^{sol} - {x}_{3} {μ}_{dop}^{sol} ) + {x}_{4} exp ( - {x}_{5} {r}_{pvc} - {x}_{6} {r}_{dop} ) - {x}_{7} exp ( - {x}_{8} {V}_{sol} ) + {x}_{9} \qquad [ Equation 1 ]$$
wherein, in Equation 1,
xₖ is each a real number from −20 to 20, and k is an integer from 1 to 9,
μᵢ^{j} is a chemical potential of the solvent, and in μᵢ^{j}, i is a solute and j is the solvent,
rᵢ is a Hansen solubility parameter distance of the solvent,
Vₛₒₗ is a molecular volume of the solvent,
sol is the solvent, and
solute is either pvc or dop, pvc is the polyvinyl chloride and dop is diethylhexyl phthalate.

11. The waste polyvinyl chloride recycled composition of claim 10, wherein the good solvent has a boiling point of 85° C. or more.

12. The waste polyvinyl chloride recycled composition of claim 10, wherein the good solvent and the non-solvent are each independently include any one of an alcohol-based solvent, a ketone-based solvent, an ester-based solvent, an ether-based solvent, a polar nonprotic solvent, a hydrocarbon solvent, or a halogen-containing solvent.

13. The waste polyvinyl chloride recycled composition of claim 10, wherein the good solvent comprises a main solvent in an amount of 50 wt % or more and an auxiliary solvent different from the main solvent in an amount of less than 50 wt %.

14. The waste polyvinyl chloride recycled composition of claim 13, wherein the main solvent of the good solvent comprises one or more of n,n-dimethylacetamide, cyclopentanone, 2-pentanone, dioxane, nitrobenzene, ethylacetate, benzene, trichloroethylene, 1,2-dichloroethane, chloroform, cyclohexanone, tetrahydrofuran, 2-butanone, acetone, or and 4-methylpentan-2-one.

15. The waste polyvinyl chloride recycled composition of claim 13, wherein the auxiliary solvent of the good solvent comprises one or more of n,n-dimethylacetamide, cyclopentanone, 2-pentanone, dioxane, nitrobenzene, ethylacetate, benzene, trichloroethylene, 1,2-dichloroethane, chloroform, cyclohexanone, tetrahydrofuran, 2-butanone, acetone, 4-methylpentan-2-one, 1,2,3-propanetrioltrinitrate, tetramethylenesulfoxide, 1-piperidinecarboxaldehyde, 1-acetylpiperidine, n-methyl-2-pyrrolidinone, 1,4-dimethylpyrrolidone, 1,3-dimethyl-2-imidazolidinone, 1,5-dimethylpyrrolidone, hexamethylphosphoramide, dimethylpropyleneurea, 1-ethyl-2-pyrrolidone, 1,3-dimethylpyrrolidone, 4-acetylmorpholine, 1-[ethoxy(methyl)phosphoryl]methylpiperidine, n,n-dimethylpropanamide, tributylphosphineoxide, 1-propyl-2-pyrrolidinone, n-isopropylpyrrolidone, n,n,4-trimethylbenzamide, n-methyl-o-acetotoluidide, 2-cyclohexen-1-one, n,n,3-trimethylbenzamide, n,n-dimethylbenzamide, trimethylenesulfide, n,n,2-trimethylbenzamide, 1,3-dioxane, tetramethylurea, 2-azacyclooctanone, 4-methyl-1-propyl-2-pyrrolidinone, 5-methyl-1-propyl-2-pyrrolidinone, n,n-diethylacetamide, 1,3-propanedithiol, 4,5-dihydro-2-methylthiazole, pyrrolidine, 3-methoxy-n,n-dimethylpropanamide, 4-fluoro-n,n-dimethylbenzamide, cycloheptanone, 1-morpholinopentan-1-one, 3-methylcyclohexanone, n,n-dimethylbutanamide, caprolactam, tetrahydrothiophene, 2-methoxy-n,n-dimethylbenzamide, dimethylformamide, 1,2-ethanedithiol, 1,4,7,10-tetraoxacyclododecane, ethyl 1,3-dimethyl-2-oxopyrrolidine-3-carboxylate, 1-(4-methylpiperidyl)butan-1-one, 2-methylcyclohexanone, 1-(2,6-dimethylmorpholin-4-yl)butan-1-one, n,n-diethylformamide, 4-(propan-2-yl)phenyldimethylcarbamate, n,n-dimethyl-3-phenylpropanamide, n-(1-methyl-2-methoxyethyl)-n-chloroacetyl-2-ethyl-6-methylaniline, dichloroacetylchloride, n,n-diethyl-3-(2-methyl-1,3-dioxolan-2-yl)propanamide, n-acetylcaprolactam, d-camphor, n-methylformanilide, ethyl n-acetyl-n-ethyl-beta-alaninate, (2,5-dimethoxyphenyl)acetaldehyde, 4-methylcyclohexanone, 2,6-dimethyl-2,5-cyclohexadiene-1,4-dione, 4-vinylcyclohexene dioxide, nicotine, 2-methylcyclopentanone, dimethylveronal, diethyl(trichloromethyl)phosphonate, 3-methyl-6-isopropylidenecyclohexanone, 1-heptanoyl-4-methylpiperazine, n,n-diethyl-4-oxopentanamide, n-ethyl-n-phenylacetamide, dimethyldisulfide, 4,6-dimethylpyrimidine, 1,4-oxathiane, n-sec-butylpyrrolidone, 1-(4-methylpiperidin-1-yl)pentan-1-one, 1-butyl-5-methyl-2-pyrrolidinone, 3-methyl-2-oxazolidone, n,n-dimethyl-4-(propan-2-yl)benzamide, 2,5-dimethylpyrazine, mesitenelactone, propyl 4-(dimethylamino)-4-oxobutanoate, diethyldimethylamidophosphate, isophorone, n,n-dimethyl 4-methoxybenzamide, 1,2,3,5-tetramethoxybenzene, 3,4,5-trimethoxytoluene, caprolactone, 3-methyl-piperidin-2-one, 2-ethylhexylsalicylate, ethyl 3-ethyl-2-oxooxolane-3-carboxylate, 1-ethyl-3,5,7-trimethyl-1,3-dihydro-azepin-2-one, dimethylisosorbide, cyclotenebutyrate, ethyl 2-(2-methoxyphenoxy)acetate, cyrene, 2,4,4-trimethyl-6-oxocyclohex-1-en-1-yl acetate, 1,2-benzodihydropyrone, tripropyleneglycolethyletherisomer, 3,4-dimethoxypropiophenone, methylaminoacetaldehydedimethylacetal, methyl 2,6-dimethoxybenzoate, 1,3-dimethyl-1,2,3,4-tetrahydro-4-quinolinone, ethyl 3,5-dimethoxybenzoate, 4-methoxybenzaldehyde, ethyl (4-methylphenoxy)acetate, 2,3,4-trimethoxyacetophenone, ethylimidazole, methyl 2-oxocyclooctanecarboxylate, ethyl 2-methyl-4-oxocyclohex-2-enecarboxylate, 2′,5′-dimethoxyacetophenone, isobutylformate, ethyl 2-(2,6-dimethylphenoxy)acetate, ethyl 2-(benzyloxy)acetate, hexylacetoacetate, diethylglutarate, 4-carbethoxy-2-ethyl-3-methyl-2-cyclohexen-1-one, nepetalactone, tripropyleneglycolbutylether, ethyl 5,5-dimethyl-2-oxofuran-3-carboxylate, 2-ethoxybenzaldehyde, methyl-2,4-dimethoxybenzoate, 5,5-diethyl-4-methoxyfuran-2(5h)-one, ethyl 4,6-dimethyl-2-oxo-3,4-dihydro-2h-pyran-5-carboxylate, ethyl 4-phenoxybutanoate, 2,5-dimethoxypropiophenone, ethyl 5,5-dimethyl-2-oxotetrahydrofuran-3-carboxylate, ethyl 3-(5-oxotetrahydrofuran-2-yl)propanoate, benzylsalicylate, n,n-diethyl-4-methyl-4h-pyridine-1-carboxamide, p-tolylacetonitrile, 5-methyl-2(3h)-furanone, 1-methyl-2-(3-pyridyl)pyrrole, formicacidpentylester, ethyl 4-(dimethylamino)benzoate, n-butylacetamide, 4-methylbenzaldehyde, 3′,4′-dimethoxyisobutyrophenone, benzaldehyde, 1,2,4-triethoxybenzene, isobutylphenoxyacetate, m-dimethoxybenzene, 3-oxobutanoicacid-1-methylethylester, ethyl2-(cyclohexylmethyl)-3-oxobutanoate, 5-ethyldihydro-2(3h)-furanone, 2-isopropyl-n,2,3-trimethylbutyramide, 4-(4-methylpiperidino)pyridine, 3-oxaspiro-undecan-2-one, formicacidhexylester, methyl 2,2-dimethyl-4,6-dioxocyclohexanecarboxylate, ethyl 6-ethyl-2-methyl-4-oxocyclohex-2-ene-1-carboxylate, benzenepropanenitrile, 4,4-dimethyl-5,6,7,8-tetrahydro-1H-isochromen-3-one, ethyl 2,6-dimethyl-4-oxocyclohex-2-enecarboxylate, n,n-dimethyloctanamide, 1-(2,5-dimethylpyrrolidin-1-yl)pentan-1-one, gamma-butyrolactone, t-butylacetoacetate, 4-(methoxymethyl)anisole, mintlactone, 2-methoxyethylacetoacetate, p-(2,2-dimethoxyethoxy)anisole, 1-(1-ethylpropyl)-2-pyrrolizinone, 2,5-dimethoxytetrahydrofuran, 2-acetoxy-1-ethoxypropane, 1,3-dimethyl-4-ethylcarbostyril, 1-(4-methoxyphenoxy)acetone, glycerol tripropionate, 3-dimethylaminoanisole, 2,4-dimethylbenzaldehyde, paraldehyde, glyceryl tributyrate, 2,3,4,5-tetramethoxytoluene, 1,3,3-trimethyl-2h-quinolin-4-one, 3-cyclohexene-1-carboxaldehyde, isobutylsalicylate, 2-(1-methylpropyl)phenyl methylcarbamate, (2,2-diethoxyethoxy)benzene, methyl 3-(5-oxooxolan-2-yl)propanoate, 4-acetoxy-2,5-dimethyl-3(2h)-furanone, 3-ethylchroman-2-one, ethyl 3-cyclohexyl-3-oxopropanoate, 1-propyl-2(1h)-pyridinone, 7-methyl-4,4a,5,6-tetrahydro-3H-naphthalen-2-one, 1-(3,5-dimethylphenoxy)propan-2-one, ethyl 3-(2,5,5-trimethyl-1,3-dioxan-2-yl)propanoate, 1-tetralone, 4-methyl-3-penten-2-one, 1-tert-butylpyrrolidin-2-one, methyl 2-(4-(tert-butyl)phenoxy)acetate, 2,6,10,10-tetramethyl-1-oxaspiro(4,5)deca-2,6-dien-8-one, ethyl 4-ethoxybenzoate, maltyl isobutyrate, 4-methyl-1,3-dioxane, 2-acetylpyridine, 1,4-diethoxybenzene, 1-(2,2-diethoxyethoxy)-4-methylbenzene, cyclohexyl lactone, 2-(2-butoxyethoxy)ethyl acetate, n-methylbenzamide, 3-acetyl-5-propyldihydrofuran-2(3h)-one, 2,2-diethoxyethoxymethylbenzene, n-methyl-m-tolylcarbamate, ethylene glycol dibutyrate, 4-cyano-2-fluorobenzoyl chloride, 3,6-dimethyl-2,3,3a,4,5,7a-hexahydro-1-benzofuran-2-one, ethyl 2-(2-oxocyclohexyl)acetate, 4-(2-acetoxyethoxy)toluene, 4-(2,5-dimethylpyrrol-1-yl)-2,6-dimethylmorpholine, 1-butylpyridin-2(1h)-one, n,n-dimethyldecanamide, valeraldehyde, 2-(3-pyridyl)piperidine, 4-methoxypropiophenone, 4-tert-butyl-n,n-dimethylbenzamide, acetylacetone, ethyl 2-methyl-2-phenoxypropanoate, ethyl 2-acetyl-5-oxohexanoate, n-sec-isopentylpyrrolidone, 4-methyl-1-tetralone, 2-butoxyethanolacetate, isobutyric anhydride, n-hexyl-2-pyrrolidinone, 3-benzyl-2,4-pentanedione, ethyl 5-oxodecanoate, methyl 2-hexyl-3-oxocyclopentanecarboxylate, 8-ethyl-1-oxaspiro[4.5]decan-2-one, n,n′-dibutylethyleneurea, methyl 3-oxododecanoate, 3-methyl-5-isopropyl-4-carbethoxy-2-cyclohexene-1-one, 6-pentyl-2h-pyran-2-one, m-methoxyacetophenone, benzisoxazole, n,n-diethylnicotinamide, ethyl 3-(2-oxocyclohexyl)propanoate, propyl 4-ethoxybenzoate, 5-butyl-5-ethyltetrahydro-2h-pyran-2-one, ethyl 2-oxo-1-propylcyclohexanecarboxylate, 2-propoxyethylacetate, dimethyl 2-methylglutarate, o-methylcorypalline, 1-pentylimidazole, n-formylmorpholine, methyl 2-butoxybenzoate, 7-butyloxepan-2-one, propanoic anhydride, acetophenone, 1-(3-methylpiperidin-1-yl)pentan-1-one, 1-butyl-2-methyl-imidazole, anisaldehyd diethyl acetal, 1-(2-methylpiperidin-1-yl)pentan-1-one, 5-butyl-5-ethyloxolan-2-one, 2,4,6-trimethylbenzaldehyde, n,n-diethylcyclohex-3-ene-1-carboxamide, butan-2-yl 4-methoxybenzoate, propylene glycol alpha methylbenzyl ether, 5-(benzyloxy)pentan-2-one, n,n-dimethyldodecanamide, n,n-diethyl-m-toluamide, (s)-dihydrobovolide, 3,4-dihydro-3-oxoedulan, butyl 2-methoxybenzoate, 7-cyclohexyloxepan-2-one, dimethyl 1,4-cyclohexanedicarboxylate, delta-decalactone, ethyl 2,5-dimethylphenoxyisobutyrate, 4a,8-dimethyl-3,4,5,6,7,8-hexahydronaphthalen-2-one, 5-hydroxy-2-decenoic acid lactone, ethyl 2-(2-oxooxolan-3-yl)acetate, methyloctalacton, 3-pentanone, 2,4-diacetylfuran, 2′-pentyloxyacetophenone, furfural, diethylmalonate, 1-(azepan-1-yl)butan-1-one, p-methoxyvalerophenone, n,n-diethylbenzamide, 4,4-dimethylnaphthalen-1(4h)-one, gamma-decalactone, piperitone, ethylacetoacetate, 1-hexylimidazole, dihydromintlactone, 4-heptanone, dimethyl glutarate, 4-(benzyloxy)-4-methylpentan-2-one, 3,3-dimethyl-1-phenoxybutan-2-one, molinate, 7-methoxy-2,2-dimethylchromene, propiophenone, ethyl 2-benzoyl-3-methylbutanoate, n,n-diethyl-2-methylcyclohexane-1-carboxamide, 5-methyl-5-propyl-1,3-dioxan-2-one, 3-methyl-5-(2-oxopropyl)oxolan-2-one, 1-(azepan-1-yl)pentan-1-one, 2-(2-ethoxyethoxy)-ethanolacetate, 2-methyl-1-(1-phenylethyl)imidazole, methyl 6-(diethylamino)-6-oxohexanoate, 5-undecanolide, 4-methyl-5-pentyloxolan-2-one, 2-ethoxyethylbenzoate, 3,4-dihydro-2(1h)-naphthalenone, 1-methoxy-3-(4-methylphenoxy)benzene, n,n-dihexylacetamide, dihydroactinidiolide, 1-pentanoylpiperidine, propan-2-yl 6-(dimethylamino)-6-oxohexanoate, 5,5-dimethyl-4-(3-oxobutyl)oxolan-2-one, 1-octylpyrrolidin-2-one, gamma-undecalactone, 4-ethoxyphenethylacetate, 2-hexanoylcyclohexan-1-one, gamma-methyldecalactone, 1-(azepan-1-yl)-3-methylbutan-1-one, methyl dihydrojasmonate, p-methoxypivalophenone, 2-acetyl-4-tert-butylcyclohexan-1-one, 4,4-dibutyl-gamma-butyrolactone, 4-ethyl-2,6-dimethoxyphenol, n,n-diethylcyclohexanecarboxamide, 5-dodecanolide, 1-hexanoylpiperidine, 1-heptyl-1h-imidazole, 2-isopropoxyethylbenzoate, butanoic anhydride, methylacetoacetate, 2,3,3-trimethylindanone, 5-hexyl-4-methyldihydrofuran-2(3h)-one, phenoxyacetone, 1-propoxy-2-propanol, ethyl di-n-propylthiolcarbamate, 1,2-dimethoxy-4-methylbenzene, tetrahydrofurfurylpropionate, 2-pyrrolidon, 2-methoxypropyl acetate, 4-dodecanolide, 2-ethoxy-4-(methoxymethyl)phenol, n-isopentylpropionanilide, pentylpiperidine-1-carboxylate, 1-(2-ethylpiperidin-1-yl)butan-1-one, isopropyl 2-isopropoxy-benzoate, n,n-dibutylpentanamide, 2,3-dihydro-2,2-dimethyl-7-hydroxybenzofuran, 2-heptanone, 2-[2-(2-methoxypropoxy)propoxy]-1-propanol, 1,2,3,4,5,6,7,8-octahydro-2,8,8-trimethylnaphthalene-2-carbaldehyde, cycloate, 4-methylquinoline, 5-nonanone, n,n-dipentylbutanamide, 3-dimethylamino-2-methylpropiophenone, 2-ethyl-1-pentanoylpiperidine, 4-(ethoxymethyl)-2-methoxyphenol, n,n-dibutylhexanamide, 2-ethylhexyllactate, tripropyleneglycolmonomethylether, magnolione, 1-octylimidazole, butylformate, butylate, 5-ethyl-2-(2-furyl)-4-propyl-m-dioxane, 1-methoxy-4-(2-methoxyethoxymethyl)benzene, hexylpiperidine-1-carboxylate, 4-propylcyclohexanone, 2,5,8,11,14,17-hexaoxaoctadecane, ethyl n,n-diethylsuccinamate, ethyl dipentylcarbamate, 2-butoxyethyl 4-methylbenzoate, 4′-pentylacetophenone, vernolate, 2-cyclopentyl cyclopentanone, 2-butoxyethylbenzoate, trimethoxymethane, phenoyethylpropionate, 4′-isobutylacetophenone, 2-methylindole, n,n,2-triethylhexanamide, 2-methoxy-4-propylphenol, 3,5-bis(2-methylpropyl)cyclohexane-1,2-dione, 2,4-diethoxy-6-methylpyrimidine, ethyl-2-hydroxybenzoate, p-ethoxybenzaldehyde, glycerol triacetate, p-hexylacetophenone, n,n-diisopropyl-phenylacetamide, 3-ethylundecane-4,6-dione, 1-benzazine, methyl 5-methylsalicylate, n,n-dipropylheptanamide, 4-methylpyridine, 4-pentylcyclohexanone, 4-pyridinecarboxaldehyde, oxolan-2-yl methylheptanoate, 2-(2-(4-methylcyclohex-3-enyl)propyl)cyclopentanone, 2-isopropoxyphenol, octanophenon, 2-benzyl-4-methyl-1,3-dioxane, ethylbenzoate, 4-methyl-2-(2-methylpropyl)oxan-4-ol, 3-(diethylamino)propiophenone, benzylacetoacetate, isoamylbenzoate, pyridine, dichloromethane, or and 1,1,2,2-tetrachloroethane.

16. The waste polyvinyl chloride recycled composition of claim 10, wherein the non-solvent comprises a main solvent in an amount of 50 wt % or more and an auxiliary solvent different from the main solvent in an amount of less than 50 wt %.

17. The waste polyvinyl chloride recycled composition of claim 16, wherein the main solvent of the non-solvent comprises one or more of propanol, diethylether, n-heptane, hexane, 1,4-butadiol, octane, cyclopentane, propyleneglycol, dimethylsulfoxide, methanol or ethanol.

18. The waste polyvinyl chloride recycled composition of claim 16, wherein the auxiliary solvent of the non-solvent comprises one or more of propanol, diethylether, n-heptane, hexane, 1,4-butadiol, octane, cyclopentane, propyleneglycol, dimethylsulfoxide, methanol, ethanol, 2,4-dimethylhexane, 3-ethyl-2,4-dimethylpentane, 2,7-dimethyloctane, 2,6-dimethyl-heptane, 2-methyl-3-ethylpentane, 3,3-dimethylhexane, 2,3,5-trimethylhexane, 2,3,4-trimethylpentane, 2,2-dimethylheptane, 3,3-dimethylpentane, 1-(perfluorobutyl)icosane, 2,5-dimethylhexane, 2,2-dimethylhexane, 2,3,3-trimethylpentane, formicacid, 2,2,6-trimethylheptane, 2,2,4-trimethylhexane, 2,2,3-trimethylpentane, 2,3,3,4-tetramethylpentane, 2,4,4-trimethylhexane, 1-hydroxybenzotriazole, 3-ethyl-2,2-dimethyl-pentane, 2,2,4,6,6-pentamethylheptane, 2,2,5-trimethylhexane, 2-nitroethanol, 2,2,3,4-tetramethylpentane, octamethyltrisiloxane, 2,2,3,3-tetramethylhexane, 1,5-pentanediol, 1,1,1,2,2,3,3-heptafluoroundecane, 2,2,5,5-tetramethylhexane, 2,2,3,3-tetramethylpentane, 2,2,4-trimethylpentane, 2,2,4,4-tetramethylpentane, dodecamethylcyclohexasiloxane, acetaldehydeammonia, 1,1,1,2,2,3,3,4,4-nonafluorohexadecane, dodecamethylpentasiloxane, 1,1,1,2,2,3,3-heptafluorononane, 1,3-dioxolan-2-one, decamethyltetrasiloxane, 1-(perfluorohexyl)docosane, hexamethyldisilane, 1,1,1,2,2,3,3,4,4,5,5-undecafluorononadecane, tetradecamethylhexasiloxane, 1,1,1,2,2,3,3,4,4,5,5,6,6-tridecafluorohexacosane, 1,1,1,2,2,3,3,4,4-nonafluoroundecane, 1,1,1,2,2,3,3,4,4-nonafluorododecane, hexadecamethylheptasiloxane, 1,1,1,2,2,3,3,4,4,5,5,6,6-tridecafluorotetracosane, propynol, 1,1,1,2,2,3,3,4,4-nonafluorodecane, ethanolamine, 1,1,1,2,2,3,3,4,4,5,5,6,6-tridecafluorodocosane, 2-hydroxypropanenitrile, 1,1,1,2,2,3,3,4,4,5,5,6,6-tridecafluoroicosane, 1,1,1,2,2,3,3,4,4-nonafluorononane, 1,1,1,2,2,3,3,4,4,5,5,6,6-tridecafluorooctadecane, cis-2-butene-1,4-diol, 1,1,1,2,2,3,3,4,4,5-decafluorononane, 1,1,1,2,2,3,3,4,4,5,5,6,6,7,7,8,8-heptadecafluorooctacosane, 1,1,1,2,2,3,3,4,4,5,5,6,6-tridecafluorohexadecane, 1,1,1,2,2,3,3,4,4,5,5,6,6,7,7,8,8-heptadecafluorohexacosane, 1,1,1,2,2,3,3,4,4,5,5,6,6,7,7,8,8-heptadecafluoropentacosane, 1,1,1,2,2,3,3,4,4,5,5,6,6-tridecafluoropentadecane, 1,1,1,2,2,3,3,4,4,5,5,6,6,7,7,8,8-heptadecafluorotetracosane, 1-(perfluorohexyl)octane, 1,1,1,2,2,3,3,4,4,5,5-undecafluorodecane, 1,1,1,2,2,3,3,4,4,5,5,6,6-tridecafluorotridecane, 1,1,1,2,2,3,3,4,4,5,5,6,6,7,7,8,8-heptadecafluorodocosane, 1h,8h-hexadecafluorooctane, 1,1,1,2,2,3,3,4,4,5,5,6,6,7,7,8,8,9,9,10,10-henicosafluorotriacontane, hexacosamethyldodecasiloxane, 1,1,1,2,2,3,3,4,4,5,5,6,6,7,7,8,8-heptadecafluoroicosane, 1,1,1,2,2,3,3,4,4,5,5,6,6-tridecafluorododecane, 1,3-propanediol, 1,1,1,2,2,3,3,4,4,5,5,6,6-tridecafluoroundecane, 1,1,1,2,2,3,3,4,4,5,5,6,6,7,7,8,8-heptadecafluorononadecane, 1,1,1,2,2,3,3,4,4,5,5,6,6,7,7,8,8,9,9,10,10-henicosafluorononacosane, 1,1,1,2,2,3,3,4,4,5,5,6,6,7,7,8,8-heptadecafluorooctadecane, 1,1,1,2,2,3,3,4,4,5,5,6,6,7,7-pentadecafluorotetradecane, 1,1,1,2,2,3,3,4,4,5,5,6-dodecafluorononane, 1,1,1,2,2,3,3,4,4,5,5-undecafluorooctane, 1,1,1,2,2,3,3,4,4,5,5,6,6,7,7-pentadecafluorotridecane, 1-perfluorooctyloctane, 1,1,1,2,2,3,3,4,4,5,5,6,6-tridecafluorodecane, 1,1,1,2,2,3,3,4,4,13,13,14,14,15,15,16,16,16-octadecafluorohexadecane, 1,1,1,2,2,3,3,4,4,5,5,6,6,7,7,8,8-heptadecafluoropentadecane, 1,1,1,2,2,3,3,4,4,5,5,6,6,7,7,8,8,9,9,10,10,11,11,12,12-pentacosafluorohentriacontane, acetamidomethanol, 1,1,1,2,2,3,3,4,4,5,5,6,6,7,7,8,8-heptadecafluorotetradecane, 1-(henicosafluorodecyl)undecane, 1,1,1,2,2,3,3,4,4,5,5,6,6,7,7,8,8-heptadecafluorotridecane, 1,1,1,2,2,3,3,4,4,5,5,6,6,7,7,8,8,9,9,10,10,11,11,12,12-pentacosafluorooctacosane, ethylenecyanohydrin, 1,1,1,2,2,3,3,4,4,5,5,6,6,7,7,8,8,9,9,10,10-henicosafluoroicosane, 1,1,1,2,2,3,3,4,4,5,5,6,6-tridecafluorooctane, perfluorooctylbromide, 1,1,1,2,2,3,3,4,4,5,5,6,6,7,7,8,8,9,9,10,10-henicosafluorooctadecane, 1,1,1,2,2,3,3,4,4,5,5,6,6,7,7,8,8-heptadecafluorododecane, 1,1,1,2,2,3,3,4,4,5,5,6,6,7,7,8,8,9,9,10,10,11,11,12,12-pentacosafluorotetracosane, pentacosafluorotricosane, 1-(perfluorohexyl)propane, 1,1,1,2,2,3,3,4,4,5,5,6,6,7,7,8,8,9,9,10,10-henicosafluoropentadecane, 1,1,1,2,2,3,3,4,4,5,5,6,6,7,7,8,8-heptadecafluorodecane, 1,1,1,2,2,3,3,4,4,5,5,6,6,7,7,8,8,9,9,10,10-henicosafluorotetradecane, 1,1,1,2,2,3,3,4,4,5,5,6,6,7,7,8,8,9,9,10,10,11,11,12,12-pentacosafluorooctadecane, 1,1,1,2,2,3,3,4,4,5,5,6,6,7,7,8,8,9,9,10,10-henicosafluorotridecane, 1,1,1,2,2,3,3,4,4,5,5,6,6,7,7,8,8,9,9,10,10-henicosafluorododecane, glycol, tetratriacontafluorotetracosane, glycerol, 1,1,1,2,2,3,3,4,4,5,5,6,6,7,7,8,8,9,9,10,10,11,11,12,12-pentacosafluorotetradecane, methylhydroperoxide, H₂O, 4-hydroxymethyl-1,3-dioxolan-2-one, hydroxyacetonitrile, perfluorooctane, perfluorodecalin, methanesulfonicacid, formamide, 4-methylphenol, PBr₃, H₂O₂, BBr₃, methylbutane, 2,2-dimethylbutane, 2,2,3-trimethyl-butane, 2,2-dimethylpentane, 2,3-dimethylbutane, pentane, 2,4-dimethylpentane, 2-methylpentane, 2-methylhexane, 3-methylpentane, 3-methylhexane, 3-ethylpentane, 3-ethyl-3-methylpentane, 2-methylheptane, 3,3-diethylpentane, 3-methylheptane, 2-methyloctane, 4-methylheptane, 3-ethylheptane, 2-methyl-nonane, 3-methyloctane, 4-methyloctane, 2-methyldecane, 3-methyl-nonane, 1,1,3,3-tetramethylcyclopentane, 1,3-butanediol, 4-methyldecane, 5-methyl-nonane, 4-methyl-nonane, 1,1-dimethylcyclopentane, 1,1,2-trimethylcyclopentane, 1,1,2-trimethylcyclohexane, n-decane, n-nonane, isobutylcyclopentane, isopropylcyclopentane, tert-butylcyclohexane, pentadecane, tridecane, 1,1,3-trimethylcyclopentane, 1,1,3-trimethylcyclohexane, nonadecane, heptadecane, octadecane, ethylcyclopentane, dodecane, n-undecane, propylcyclopentane, isobutylcyclohexane, isopropylcyclohexane, 1-ethyl-1-methylcyclopentane, 2,3,3-trimethyl-1-butene, 2,4,4-trimethyl-1-pentene, n-hexylcyclopentane, pentylcyclopentane, 1-isopropyl-4-methylcyclohexane, trimethylaluminum, n-tetradecylcyclopentane, tridecylcyclopentane, tetradecane, n-dodecylcyclopentane, n-decylcyclopentane, n-propylcyclohexane, ethylcyclohexane, 3,3-dimethyl-1-butene, n-hexadecane, pentene, 2,3,3-trimethyl-1-pentene, decylcyclohexane, pentylcyclohexane, 3,4-dicyclohexyl-2,5-dimethylhexane, 2-methyl-1-butene, 3,3-dimethyl-1-pentene, 4,4-dimethyl-1-pentene, butylcyclohexane, methylcyclopentane, methylcyclohexane, decahydronaphthalene, hexahydroindene, bicyclohexyl, cyclohexane, 2,4,4-trimethyl-2-pentene, 2,3-dimethyl-1-pentene, tricyclohexylmethane, butylcyclopentane, 1-hexene, 2,3-dimethyl-1-butene, methylformamide, 3,3-dimethyl-1-butyne, triethyleneglycol, 3-methoxy-1,2-propanediol, 2,4-dimethyl-1-pentene, 1-heptene, 3-methyl-1-pentene, 3-ethyl-2-methyl-1-pentene, 1,4-di-tert-butylbenzene, diethyleneglycol, methoxyaceticacid, 1-nonene, 4-methyl-1-hexene, 1,1,3,4,4,6-hexamethyltetralin, 1,3,5-triisopropylbenzene, 1,2,2,6,6-pentamethylpiperidine, 1,4-diethylbenzene, 2-methyl-2-butene, isoprene, 4-methyl-1-pentene, 1-tetradecene, 3-ethyl-1-pentene, 2-methyl-3-heptene, 1-decene, 2,3,4-trimethyl-2-pentene, 1-nonadecene, butylbenzene, 2-ethoxy-2-methyl-propane, 1,2-diethylbenzene, ethanedial, cyclohexylbenzene, galaxolide, 1,2,3,4-tetramethylbenzene, 1,2,3,5-tetramethylbenzene, pentaethylbenzene, 1,3,5-trimethylbenzene, isobutanol, peroxyaceticacid, 1,6-hexanediol, 2,6-di-tert-butyl-4-methylpyridine, 2,2′-(methylimino)bis-ethanol, 1,3-bis(1-methylethyl)-benzene, 2,7-diisopropylnaphthalene, 1,2,3,5-tetraethylbenzene, tetrahydro-2,2,5,5-tetramethylfuran, 1,5-diisopropylnaphthalene, 2,3-butanediol, 1,2-diisopropylbenzene, 1-(2-pentyl)-4-(3-pentyl)benzene, tetradecylbenzene, pentadecylbenzene, ethylbenzene, 3,6-bis-(methoxymethyl)-durene, 5-hydroxy-6-methoxy-8-[(4-amino-1-methylbutyl)amino]quinoline trihydrobromide, 1-(1,1-dimethylethyl)-3-methylbenzene, tridecylbenzene, 1-(methoxymethyl)-4-(2,4,4-trimethylpentan-2-yl)benzene, dodecylbenzene, 1,2,4-trimethylbenzene, 1-cyclohexyl-2-isopropylbenzene, 4,6,6-Trimethylbicyclo(3,1,1)hept-3-ene, 2,6-di-tert-butylpyridine, 1,1′-oxybis-octane, 1,5-hexanediol, 1-(1,1-dimethylethyl)-3,5-dimethylbenzene, menthylisovalerate, 1-methyl-7-(1-methylpropyl)naphthalene, tert-butanol, beta-himachalen, dimethoxymethane, 1,2,3,4,5,6,7,8-octahydroanthracene, tert-butylbenzene, 1,2-hexanediol, cyclohexene, 6,10,14-trimethylpentadecan-2-one, delta-cadinene, isopropylbenzene, undecylbenzene, 2,5-hexanediol, dipropyleneglycol, 1,6-dimethyl-4-isopropyltetralin, di-n-propylether, diisopropylether, cuparene, 1,4-bis(1-methylethyl)-benzene, 2,4-dimethyl-3-thiapentane, 1-methoxy-4-(2,4,4-trimethylpentan-2-yl)benzene, 1-ethoxybutane, decylbenzene, or cycloheptane.
